(19) United States
(12) Patent Application Publication
(10) Pub. No.: US 2003/0049246 A1
(43) Pub. Date: Mar. 13, 2003

(54) Gli protein truncate and uses thereof

(76) Inventor: Ariel Ruiz I Altaba, New York, NY (US)

Correspondence Address: KLAUBER & JACKSON, 411 HACKENSACK AVENUE, HACKENSACK, NJ, 07601

(21) Appl. No.: 10/139,092

(22) Filed: May 3, 2002

Related U.S. Application Data
(63) Continuation of application No. 09/371,379, filed on Aug. 10, 1999, now abandoned.
(60) Provisional application No. 60/096,129, filed on Aug. 10, 1998.

(51) Int. Cl. A61K 38/47; C07H 21/04; C12N 9/22; C12P 21/02; C12N 5/08

(52) U.S. Cl. 424/94.61; 435/69.1; 435/320.1; 435/199; 435/368; 536/23.2

(57) ABSTRACT

This invention provides an isolated protein comprising an amino acid sequence of a C-terminal Gli 1 truncate, wherein the protein acts as a dominant-negative repressor of neuronal differentiation. This invention provides an isolated protein comprising an amino acid sequence of a C-terminal Gli 2 truncate, wherein the protein acts as a dominant-negative repressor of neuronal differentiation. This invention provides an isolated protein comprising an amino acid sequence of a C-terminal Gli 3 truncate, wherein the protein acts as a dominant-negative repressor of neuronal differentiation. In addition, this invention provides a pharmaceutical composition comprising an amount of the protein truncate or fragment and a pharmaceutically acceptable carrier ordiluent. This invention provides a method of identifying a test composition or agent which modulates Gli 1, Gli 2, or Gli 3 and a method of identifying/screening a cell for protein truncates or fragments of Gli protein family. Lastly, this invention provides a method for treating a subject having Polydactyly Type A (PAP-A) or Pallister-Hall Syndrome (PHS).

### CROSS-REFERENCE TO RELATED APPLICATIONS

The present application is a Continuation of U.S. patent application Ser. No. 09/371,379, filed Aug. 10, 1999, which claims priority to provisional U.S. application Serial No. 60/096,129, filed on Aug. 10, 1998, the disclosures of which are incorporated by reference herein in their entireties.

### GOVERNMENT RIGHTS CLAUSE

The research leading to the present invention was supported, at least in part, by grants from The National Institutes of Health (CA34610, and NS37352). Accordingly, the Government may have certain rights in the invention.

### FIELD OF THE INVENTION

This invention provides an isolated protein comprising an amino acid sequence of a C-terminal Gli 1 truncate, wherein the protein acts as a dominant-negative repressor of neuronal differentiation. This invention provides an isolated protein comprising an amino acid sequence of a C-terminal Gli 2 truncate, wherein the protein acts as a dominant-negative repressor of neuronal differentiation. This invention provides an isolated protein comprising an amino acid sequence of a C-terminal Gli 3 truncate, wherein the protein acts as a dominant-negative repressor of neuronal differentiation. This invention provides a method of identifying/screening a cell for protein truncates or fragments of Gli protein family. Lastly, this invention provides a method for treating a subject having Polydactyly Type A (PAP-A) or Pallister-Hall Syndrome (PHS).

### BACKGROUND OF THE INVENTION

How cells interpret Hedgehog signaling is key to understanding many patterning events in the early vertebrate embryo. Genetic and molecular analyzes have drawn a pathway for the transduction of the Hedgehog signals in which a receptor complex acts through a series of intermediate cytoplasmic steps to activate the function of Gli family zinc finger transcription factors (Forbes et al., 1993, Dev. Suppl. 115-124). Analyzes in Drosophila have shown that Cubitus interruptus (Orenic et al., 1990, Drosophila Genes & Dev. 4:1053-1067; Eaton and Kornberg, 1990, Genes & Dev. 4: 1068-1077), a Gli family member, is necessary and sufficient to mediate Hedgehog signaling (Alexandre et al., 1996, Genes & Dev. 10: 2003-2013; Dominguez et al., 1996, Science 272: 1621-1625; Hepker et al.1997, Development 124: 549-558; von Ohnen et al.1997, PNAS 94: 2404-2409). Regulation of Ci is complex as it associates with other proteins docking at microtubules even though it acts as a nuclear transcription factor (Aza-Blanc et al.1997, Cell 89:1043-1053; Robbins et al., 1997, Cell 90:225-234; Sisson et al., 1997, Cell 90:235-245),

In vertebrates, three Gli genes have been described in several species (Kinzler et al., 1987, Science 236: 70-73; Ruppert et al., 1988, Mol. Cell Biol. 8: 3104-3113; Ruppert et al.,1990 Mol. Cell Biol. 10: 5408-5415; Walterhouse et al., 1993, Develop. Dyn. 196: 91-102; Hui et al., 1994, Develop. Biol. 162: 402-413; Marigo et al., 1996, Dev. Biol. 180: 273-283; Lee et al., 1997, Development, 124: 2537-2552; Marine et al., 1997, Mech. Dev. 63: 211-225; Hughes et al., 1997, Genomics 39: 205-215) The first Gli gene (hereafter Gli1) was isolated from a glioma line (Kinzler et al., 1987, Science 236: 70-73) and is an oncogene (Ruppert et al., 1991, Mol. Cell Biol. 11: 1724-1728; Dahmane et al., 1997, Nature 389: 876-881) possibly harboring a mutation (Hynes et al., 1997, Neuron 19: 15-26). The vertebrate Gli genes have different functions in embryonic development (Lee et al., 1997, Development 1124: 2537-2552; Mo et al., 1997, Development 124: 113-123; Sasaki et al., 1997, Development 124: 1313-1322; Ruiz i Altaba, 1998, Development 125: 2203-2212) and are transcriptionally (Marigo et al., 1996, Dev. Biol. 180: 273-283; Lee et al., 1997, Development 1124: 2537-2552; Sasaki et al., 1997, Development 124: 1313-1322; Ruiz i Altaba, 1998, Development 125: 2203-2212) and possibly translationally (Jan et al., 1997, EMBO J. 16: 6301-6313) regulated in different ways. Both Gli1 (Marigo et al., 1996, Dev. Biol. 180: 273-283; Lee et al., 1997, Development 124: 2537-2552) and Gli2 (Ruizi Altaba, 1998, Development 125: 2203-2212) are targets of Sonic hedgehog (Shh) signaling whereas Gli3 and Shh show a mutually repressive interaction (Masuya et al., 1995, Dev. Biol. 182: 42-51; Marigo et al., 1996, Dev. Biol. 180: 273-283; Byscher et al., 1997, Mech. Dev. 62: 175-182; Ruiz i Altaba, 1998, Development 125: 2203-2212).

Consistent with this, in the early frog neural plate Gli1 is expressed in midline and immediately adjacent cells close to the underlying Shh-secreting notochord, Gli2 is expressed throughout the neural plate with the exception of the midline and Gli3 is also absent from the midline but shows graded distribution with highest levels laterally (Lee et al., 1997, Development 124: 2537-2552). Only Gli1 can induce floor plate differentiation (Lee et al., 1997, Development 124: 2537-2552; Sasaki et al., 1997, Development 124: 1313-1322; Hynes et al., 1997, Neuron 19: 15-26; Ruiz i Altaba, 1998, Development 125: 2203-2212) but all three Gli genes can induce neuronal development (Brewster et al., 1998, Nature, in press). However, each Gli gene appears to induce different sets of neurons (Ruiz i Altaba, 1998, Development 125: 2203-2212).

These and other results on skeletal patterning (Mo et al., 1997, Development 124: 113-123) indicate that there is functional divergence but perhaps also partial redundancy in the Gli gene family and provide support for the idea that the overall Gli readout of a cell is instructive in determining its fate (Ruiz i Altaba, 1997, Cell 193-196). However, it remains unknown how Gli proteins act, especially since there is the possibility that, like Ci in Drosophila, Glis are processed to yield different forms with varying activities: Ci is cleaved to yield a repressor lacking most of the C -terminal sequences and cleavage is repressed by Hedgehog signaling (Aza-Blanc et al., 1997, Cell 89: 1043-1053).

### SUMMARY OF THE INVENTION

This invention provides an isolated protein comprising an amino acid sequence of a C-terminal Gli 1 truncate, wherein the protein acts as a dominant-negative repressor of neuronal differentiation. This invention provides an isolated protein comprising an amino acid sequence of a C-terminal Gli 2 truncate, wherein the protein acts as a dominant-negative repressor of neuronal differentiation. This invention provides an isolated protein comprising an amino acid sequence of a C-terminal Gli 3 truncate, wherein the protein acts as a dominant-negative repressor of neuronal differentiation.

This invention provides an isolated C-terminal Gli 1 truncate protein, wherein the protein comprises 1075-1125 amino acids and acts as a dominant-negative repressor of neuronal differentiation. This invention provides an isolated C-terminal Gli 1 truncate protein, wherein the protein comprises 735-785 amino acids and acts as a dominant-negative repressor of neuronal differentiation. This invention provides isolated C-terminal Gli 1 truncate protein, wherein the protein comprises 515-565 amino acids and acts as a dominant-negative repressor of neuronal differentiation. This invention provides isolated C-terminal Gli 3 truncate protein, wherein the protein comprises 975-1025 amino acids and acts as a dominant-negative repressor of neuronal differentiation. This invention provides an isolated C-terminal Gli 3 truncate protein, wherein the protein comprises 865-915 amino acids and acts as a dominant-negative repressor of neuronal differentiation. This invention provides an isolated C-terminal Gli 3 truncate protein, wherein the protein comprises 735-785 amino acids and acts as a dominant-negative repressor of neuronal differentiation. This invention provides an isolated C-terminal Gli 3 truncate protein, wherein the protein comprises 775-725 amino acids and acts as a dominant-negative repressor of neuronal differentiation. This invention provides isolated C-terminal Gli 3 truncate protein, wherein the protein comprises 620-670 amino acids and acts as a dominant-negative repressor of neuronal differentiation.

This invention provides a pharmaceutical composition comprising an amount of the protein truncate or fragment and a pharmaceutically acceptable carrier or diluent.

This invention provides a method of testing the ability of a drug, agent, or compound to modulate the activity of the protein truncate or fragment as hereinabove disclosed, which comprises: (a) culturing test cells which contain elevated levels of the protein truncate or fragment in a tumorous condition; (b) adding the drug, agent, or compound under test; and (c) measuring the change if any, in the tumorous condition of said test cells.

This invention provides a method for identifying a test composition or agent which modulates Gli 1, Gli 2, or Gli 3 proteins or any other Gli family protein which comprises: (a) contacting a protein truncate or fragment as hereinabove disclosed with a test composition or agent under conditions permitting binding between the protein and the test composition; (b) detecting specific binding of the a test composition or agent to the proteins; and (c) determining whether the a test composition or agent inhibits the proteins, so as to identify a test composition or agent which is which modulates Gli 1, Gli 2, or Gli 3.

This invention provides a method of identifying a test composition or agent which modulates Gli 1, Gli 2, or Gli 3 or any other Gli protein family, the method comprising: (a) incubating components comprising the test composition, and the proteins, wherein the incubating is carried out under conditions sufficient to permit the components to interact; and (b) measuring the effect of the test composition on the binding to the proteins.

This invention provides a method of identifying/screening a cell for protein truncates or fragments of Gli protein family comprising, introducing into the cell the protein truncates or fragments, wherein the protein inhibits the function or activity of a protein of the Gli family; and detecting the resulting protein produced, thereby identifying/screening the cell for protein truncates or fragments of the Gli protein family. Gli family proteins include but are not limited to; Gli 1, Gli 2, Gli 3 and other homologous proteins.

This invention provides a method of inhibiting the function, or processing of Gli 1 or Gli 3, comprising introducing into a cell the protein truncates or fragments as hereinabove disclosed, or the vector comprising the nucleic acid encoding the protein truncates or fragments as hereinabove disclosed, thereby inhibiting the function of or processing of Gli 1, Gli 2, or Gli 3 protein.

This invention provides a method for treating a subject having Polydactyly Type A (PAP-A) or Pallister-Hall Syndrome (PHS), comprising administering a therapeutically effective amount of the pharmaceutical composition comprising the protein truncates or fragments as hereinabove disclosed, or the vector comprising the nucleic acid encoding the protein truncates or fragments as hereinabove disclosed, so as to inhibit the function or processing of Gli 1 or Gli 3 expression, thereby treating the subject having Polydactyly Type A (PAP-A) or Pallister Syndrome (PHS).

### BRIEF DESCRIPTION OF THE DRAWINGS

FIG. 1. Diagramatic summary of results. Putative processing sites are shown as arrows with their approximate locations (+/−25 aa) given on top of each arrow in aa. Large arrows point to major sites and small arrows to minor sites determine by the relative abundance of the appropriate products. Each box represents a Gli protein. Amino-acid numbers are given under each protein. Myc-tags are shown as black dots at the amino termini. Conserved regions are according to Ruppert et al. (1990, Mol. Cell Biol. 10: 5408-5415) and are depicted by small lines over the protein diagram with their corresponding number. Amino acid numbers for fGli2 (asterisks) correspond to the sequence of Marine et al. (1997, Mech. Dev. 63: 211-225). The cDNA used here is allellic (Brewster et al., 1998, Nature, in press). Black dots inside boxes are putative PKA phosphorylation sites.

FIG. 2. Diagram of full-length and deletion forms of Gli proteins and summary of localization and results of functional assays. The name of the constructs is given to the left. The bar diagrams show the structure of the different Gli proteins in the middle. The zinc finger domain is shown in black and all proteins are aligned according to this domain. Sizes and sites of truncation are shown in amino acid numbers. Conserved domains are depicted above the protein bar diagrams as dashes with their appropriate number according to Ruppert et al. (1990, Mol. Cell Biol. 10: 5408-5415). Region 2 includes the zinc finger domain. Arrows point to major (large arrows) and minor (small arows) processing sites. Internal deletions are shown connected by diagonal lines (e.g. in hGli3CΔSmaI). Localization (Loc) of protein products in also represented as nuclear (N, n) and/or cytoplasmic (C, c) with lower case indicating minor localization in the compartment. The presence (+) or absence (−) of activity in activation (Act) or repression (Rep) of HNF−3β or N-tubulin (N-tub) of the different constructs is also shown in the right columns. Absence of a symbol represents experiments not done.

FIG. 3. Schematic diagram of the structure and functional domains of Gli family proteins. Proteins are depicted as horizontal boxes with N- and C-terminal ends indicated. The Myc tags are represented by black circles in the amino terminus. The zinc finger (ZF) domain is shown in black. Arrows point to approximate processing sites. Black dots inside boxes are consensus PKA phosphorylation sites. Conserved domains are numbered and shown by a horizontal line over the boxes. Brackets depict the approximate localization of different domains. Brackets in Tra1 show the clustering of mutations that either activate or inactivate Tra1 function. Inactivating mutations cluster in the equivalent of region 3, where the PAP-A and PHS mutations are located in Gli3. In both cases, the severity of the mutations (triangle in Tra1) increases the closer they are to the zinc finger domain. Activating mutations map to the N-terminus which encodes a repressor function in Glis. These observations suggest a conservation of structural features important for function.

FIG. 4: Activation and repression assay.

FIG. 5. Repressor forms of Gli3, but not Gli1, bind Smads: a) Schematic diagram of Gli1, Gli3, a C-terminal deletion of Gli3 (Gli3C′ΔClaI), and a similar C-terminal deletion of Gli1 (Gli1C′ΔPstI). The two deletions mimic processed forms¹¹. Conserved regions in Gli proteins are denoted by the corresponding number. ZF refers to the five zinc finger domain shown in a black box. The circle at the N-terminus represents Myc epitopes. Numbers refer to aa in the protein sequences.

FIG. 6. The last three zinc fingers of Gli3 are required for binding to Smad1. a) Schematic representation of Gli3 and deletion derivatives similar to that in FIG. 3a. Sites of truncation are denoted by the amino acid number. Bars show the sites of frame-shift mutations causing Polydactyly type A (PAP-A) and Pallister-Hall syndrome (PHS). The column to the right summarizes the results shown in (b) depicting the ability (+) or inability (−) of the various Gli3 proteins to bind Smads.

### DETAILED DESCRIPTION OF INVENTION

As demonstrated herein, the formation of distinct products from injected tagged-Gli constructs into frog embryos has been analyzed and Gli protein structure correlated with a floor plate and neuronal inductions. The results show that Gli genes yield proteins of different sizes encoding distinct functions. Each Gli gene has a distinct pattern of protein products that are, nevertheless, similar. The N-terminal region encodes a repressor function whereas C-terminal regions are required for positive inducing activity in different assays. Repressors are nuclear and act as dominant-negative forms. Activating constructs, in contrast, appear to be mostly cytoplasmic. The importance of repressor derivatives is highlighted by the similarity in structure of two forms of Gli3 with frame-shift mutants that terminate translation prematurely in humans. Consistent with the dominant-negative effects of the repressor forms, the human mutations have been found to act dominantly. In addition, the present results demonstrate that endogenous Gli proteins are processed to yield different forms with distinct activities the production of which may be regulated by inductive signaling.

This invention provides an isolated protein comprising an amino acid sequence of a C-terminal Gli 1 truncate, wherein the protein acts as a dominant-negative repressor of neuronal differentiation. This invention provides an isolated protein comprising an amino acid sequence of a C-terminal Gli 2 truncate, wherein-the protein acts as a dominant-negative repressor of neuronal differentiation. This invention provides an isolated protein comprising an amino acid sequence of a C-terminal Gli 3 truncate, wherein the protein acts as a dominant-negative repressor of neuronal differentiation.

The isolated protein comprising an amino acid sequence of a C-terminal Gli 1 truncate may also have floor plate inducing activity and other activities associates with Gli 2 and 3 proteins. For example, other Gli associates activities, include but are not limited to: mesodermal development, and muscle and bone formation.

As defined herein “dominant-negative repressor” means the ability of a protein truncate to inhibit the function or activity of a full-length protein of the Gli family, such as Gli 1, Gli 2, or Gli 3.

In one embodiment the isolated protein is a C-terminal deleted Pst1 Gli 1 truncate. The isolated protein is made by cleaving the nucleic acid sequence which codes for an amino acid sequence of a full-length Gli 1 protein with a PstI restriction endonuclease, thereby creating a C-terminal deleted Pstl Gli 1 truncate. In another embodiment the isolated protein has the amino acid sequence as set forth in SEQ ID NO:1, including fragments, mutants, and variants thereof. In the preferred embodiment the protein has the amino acid sequence from amino acid position 1 to position 515: SEQ ID NO:1.

In one embodiment the isolated protein is a C-terminal deleted BsaBI Gli 1 truncate. The isolated protein is made by cleaving the nucleic acid sequence which codes for an amino acid sequence of a full-length Gli 1 protein with a BsaBI restriction endonuclease, thereby creating a C-terminal deleted BsaBI Gli 1 truncate. In another embodiment the isolated protein has the amino acid sequence as set forth in SEQ ID NO:2, including fragments, mutants, and variants thereof. In the preferred embodiment the protein has the amino acid sequence from amino acid position 1 to position 1253: SEQ ID NO:2.

In one embodiment the isolated protein is a C-terminal deleted Agel Gli 1 truncate. The isolated protein is made by cleaving the nucleic acid sequence which codes for an amino acid sequence of a full-length Gli 1 protein with an Agel restriction endonuclease, thereby creating a C-terminal deleted Agel Gli 1 truncate. In another embodiment the isolated protein has the amino acid sequence as set forth in SEQ ID NO:3, including fragments, mutants, and variants thereof. In the preferred embodiment the protein has the amino acid sequence from amino acid position 1 to position 525: SEQ ID NO:3.

In one embodiment the isolated protein is a C-terminal deleted XhoI Gli 3 truncate. The isolated protein is made by cleaving the nucleic acid sequence which codes for an amino acid sequence of a full-length Gli 3 protein with an XhoI restriction endonuclease, thereby creating a C-terminal deleted XhoI Gli 3 truncate. In another embodiment the isolated protein has the amino acid sequence as set forth in SEQ ID NO:4, including fragments, mutants, and variants thereof. In the preferred embodiment the protein has the amino acid sequence from amino acid position 1 to position 557: SEQ ID NO:4.

In one embodiment the isolated protein is a C-terminal deleted Bal#8 Gli 3 truncate. The isolated protein is made by cleaving the nucleic acid sequence which codes for an amino acid sequence of a full length Gli 3 protein with a Bal#8 restriction endonuclease, thereby creating a C-terminal deleted Bal#8 Gli 3 truncate. In another embodiment the isolated protein has the amino acid sequence as set forth in SEQ ID NO:5, including fragments, mutants, and variants thereof. In the preferred embodiment the protein has the amino acid sequence from amino acid position 1 to position 700: SEQ ID NO:5.

In one embodiment the isolated protein is a C-terminal deleted ClaI Gli 3 truncate. The isolated protein is made by cleaving the nucleic acid sequence which codes for an amino acid sequence of a full-length Gli 3 protein with a ClaI restriction endonuclease, thereby creating a C-terminal deleted ClaI Gli 3 truncate. In another embodiment the isolated protein has the amino acid sequence as set forth in SEQ ID NO:6, including fragments, mutants, and variants thereof. In the preferred embodiment the protein has the amino acid sequence from amino acid position 1 to position 745: SEQ ID NO:6.

This invention provides an isolated C-terminal Gli 1 truncate protein, wherein the protein comprises 1075-1125 amino acids beginning at position 1 of the amino acid sequence and acts as a dominant-negative repressor of neuronal differentiation. In one embodiment the amino acid sequence is set forth in SEQ ID NO:7. This invention provides an isolated C-terminal Gli 1 truncate protein, wherein the protein comprises 735-785 amino acids beginning at position 1 of the amino acid sequence as set forth in FIG. 11 and acts as a dominant-negative repressor of neuronal differentiation. In one embodiment the amino acid sequence is set forth in SEQ ID NO: 8. This invention provides isolated C-terminal Gli 1 truncate protein, wherein the protein comprises 515-565 amino acids beginning at position 1 of the amino acid sequence and acts as a dominant-negative repressor of neuronal differentiation. In one embodiment the amino acid sequence is set forth in SEQ ID NO:9. This invention provides isolated C-terminal Gli 3 truncate protein, wherein the protein comprises 975-1025 amino acids beginning at position 1 of the amino acid sequence and acts as a dominant-negative repressor of neuronal differentiation. In one embodiment the isolated protein, wherein the amino acid sequence is set forth in SEQ ID NO:10. This invention provides an isolated C-terminal Gli 3 truncate protein, wherein the protein comprises 865-915 amino acids beginning at position 1 of the amino acid sequence and acts as a dominant-negative repressor of neuronal differentiation. In one embodiment isolated protein, wherein the amino acid sequence is set forth in SEQ ID NO: 11. This invention provides an isolated C-terminal Gli 3 truncate protein, wherein the protein comprises 735-785 amino acids beginning at position 1 of the amino acid sequence and acts as a dominant-negative repressor of neuronal differentiation. In one embodiment the isolated protein, wherein the amino acid sequence is set forth in SEQ ID NO:12. This invention provides an isolated C-terminal Gli 3 truncate protein, wherein the protein comprises 775-725 amino acids beginning at position 1 of the amino acid sequence and acts as a dominant-negative repressor of neuronal differentiation. In one embodiment the amino acid sequence is set forth in SEQ ID NO:13. This invention provides isolated C-terminal Gli 3 truncate protein, wherein the protein comprises 620-670 amino acids beginning at position 1 of the amino acid sequence and acts as a dominant-negative repressor of neuronal differentiation. In one embodiment the amino acid sequence is set forth in SEQ ID NO: 14.

This invention is directed to analogs of the protein which comprise the amino acid sequence as set forth above. The analog protein may have an N-terminal methionine or an N-terminal polyhistidine optionally attached to the N or COOH terminus of the protein which comprise the amino acid sequence.

In another embodiment, this invention contemplates peptide fragments of the protein which result from proteolytic digestion products of the protein. In another embodiment, the derivative of the protein has one or more chemical moieties attached thereto. In another embodiment the chemical moiety is a water soluble polymer. In another embodiment the chemical moiety is polyethylene glycol. In another embodiment the chemical moiety is mon-, di-, tri- or tetrapegylated. In another embodiment the chemical moiety is N-terminal monopegylated.

Attachment of polyethylene glycol (PEG) to compounds is particularly useful because PEG has very low toxicity in mammals. Numerous activated forms of PEG suitable for direct reaction with proteins have been described. Useful PEG reagents for reaction with protein amino groups include active esters of carboxylic acid or carbonate derivatives, particularly those in which the leaving groups are N-hydroxysuccinimide, p-nitrophenol, imidazole or 1-hydroxy-2-nitrobenzene-4-sulfonate. PEG derivatives containing maleimido or haloacetyl groups are useful reagents for the modification of protein free sulfhydryl groups. Likewise, PEG reagents containing amino hydrazine or hydrazide groups are useful for reaction with aldehydes generated by periodate oxidation of carbohydrate groups in proteins.

In one embodiment, the amino acid residues of the protein described herein are preferred to be in the “L” isomeric form. In another embodiment, the residues in the “D” isomeric form can be substituted for any L-amino acid residue. NH₂ refers to the free amino group present at the amino terminus of a protein. COOH refers to the free carboxy group present at the carboxy terminus of a protein. Abbreviations used herein are in keeping with standard protein nomenclature, J Biol. Chem., 243:3552-59 (1969).

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. Synthetic protein, prepared using the well known techniques of solid phase, liquid phase, or peptide condensation techniques, or any combination thereof, can include natural and unnatural amino acids. Thus, protein of the invention may comprise D-amino acids, a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps, α-helices, β turns, β sheets, γ-turns, and cyclic peptides can be generated. In an additional embodiment, pyroglutamate may be included as the N-terminal residue of the peptide.

The present invention further provides for modification or derivatization of the protein of the invention. Modifications of proteins are well known to one of ordinary skill, and include phosphorylation, carboxymethylation, and acylation. Modifications may be effected by chemical or enzymatic means. In another aspect, glycosylated or fatty acylated peptide derivatives may be prepared. Preparation of glycosylated or fatty acylated peptides is well known in the art. Fatty acyl peptide derivatives may also be prepared. For example, and not by way of limitation, a free amino group (N-terminal or lysyl) may be acylated, e.g., myristoylated. In another embodiment an amino acid comprising an aliphatic side chain of the structure —(CH₂)ₙCH₃ may be incorporated in the peptide. This and other peptide-fatty acid conjugates suitable for use in the present invention are disclosed in U.K. Patent GB-8809162.4, International Patent Application PCT/AU89/00166, [and reference 5, supra].

Mutations can be made in a nucleic acid encoding the protein such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycinc, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Synthetic DNA sequences allow convenient construction of genes which will express analogs or “muteins”. A general method for site-specific incorporation of unnatural amino acids into proteins is described in Noren, et al. Science, 244:182-188 (April 1989). This method may be used to create analogs with unnatural amino acids.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Chemical moieties suitable for derivatization may be selected from among water soluble polymers. The polymer selected should be water soluble so that the component to which it is attached does not precipitate in an aqueous environment, such as a physiological environment. Preferably, for therapeutic use of the end-product preparation, the polymer will be pharmaceutically acceptable. One skilled in the art will be able to select the desired polymer based on such considerations as whether the polymer/component conjugate will be used therapeutically, and if so, the desired dosage, circulation time, resistance to proteolysis, and other considerations. For the present component or components, these may be ascertained using the assays provided herein.

The water soluble polymer may be selected from the group consisting of, for example, polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols and polyvinyl alcohol. Polyethylene glycol propionaldenhyde may have advantages in manufacturing due to its stability in water.

This invention provides an isolated nucleic acid fragment which encodes an amino acid sequence of a C-terminal Gli 1 truncate, wherein the protein acts as a dominant-negative repressor of neuronal differentiation. In one embodiment the nucleic acid is a C-terminal deleted Pstl Gli 1 fragment. In another embodiment the nucleic acid is a C-terminal deleted BsaBI Gli 1 fragment. In another embodiment the nucleic acid is a C-terminal deleted Agel Gli 1 fragment.

This invention provides an isolated nucleic acid fragment which encodes an amino acid sequence of a C-terminal Gli 2 truncate, wherein the protein acts as a dominant-negative repressor of neuronal differentiation.

This invention provides an isolated nucleic acid fragment which encodes an amino acid sequence of a C-terminal Gli 3 truncate, wherein the protein acts as a dominant-negative repressor of neuronal differentiation. In one embodiment the nucleic acid is a C-terminal deleted XhoI Gli 3 fragment. In another embodiment the nucleic acid is a C-terminal deleted Bal#8 Gli 3 fragment. The nucleic acid is DNA, cDNA, genomic DNA, RNA. Further, the isolated nucleic acid may be operatively linked to a promoter of RNA transcription.

A “vector” is a replicon, such as plasmid, phage or cosmid, to which another DNA segment may be attached so as to bring about the replication of the attached segment.

A “DNA” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

Further this invention also provides a vector which comprises the above-described nucleic acid molecule. The promoter may be, or is identical to, a bacterial, yeast, insect or mammalian promoter. Further, the vector may be a plasmid, cosmid, yeast artificial chromosome (YAC), bacteriophage or eukaryotic viral DNA.

Other numerous vector backbones known in the art as useful for expressing protein may be employed. Such vectors include, but are not limited to: adenovirus, simian virus 40 (SV40), cytomegalovirus (CMV), mouse mammary tumor virus (MMTV), Moloney murine leukemia virus, DNA delivery systems, i.e. liposomes, and expression plasmid delivery systems. Further, one class of vectors comprises DNA elements derived from viruses such as bovine papilloma virus, polyoma virus, baculovirus, retroviruses or Semliki Forest virus. Such vectors may be obtained commercially or assembled from the sequences described by methods well-known in the art.

This invention also provides a host vector system for the production of a protein which comprises the vector of a suitable host cell. Suitable host cells include, but are not limited to, prokaryotic or eukaryotic cells, e.g. bacterial cells (including gram positive cells), yeast cells, fungal cells, insect cells, and animals cells. Numerous mammalian cells may be used as hosts, including, but not limited to, the mouse fibroblast cell NIH 3T3, CHO cells, HeLa cells, Ltk⁻ cells, Cos cells, etc.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage A, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

This invention further provides a method of producing a protein which comprises growing the above-described host vector system under suitable conditions permitting the production of the protein and recovering the protein so produced.

This invention further provides an antibody capable of specifically recognizing or binding to the isolated protein. The antibody may be a monoclonal or polyclonal antibody. Further, the antibody may be labeled with a detectable marker that is either a radioactive, calorimetric, fluorescent, or a luminescent marker. The labeled antibody may be a polyclonal or monoclonal antibody. In one embodiment, the labeled antibody is a purified labeled antibody. Methods of labeling antibodies are well known in the art.

The term “antibody” includes, by way of example, both naturally occurring and non-naturally occurring antibodies. Specifically, the term “antibody” includes polyclonal and monoclonal antibodies, and fragments thereof. Furthermore, the term “antibody” includes chimeric antibodies and wholly synthetic antibodies, and fragments thereof. Such antibodies include but are not limited to polyclonal, monoclonal, chimeric, single chain, Fab fragments, and an Fab expression library.

Various procedures known in the art may be used for the production of polyclonal antibodies to protein or derivatives or analogs thereof (see, e.g., Antibodies—A Laboratory Manual, Harlow and Lane, eds., Cold Spring Harbor Laboratory Press: Cold Spring Harbor, N.Y., 1988). For the production of antibody, various host animals can be immunized by injection with the truncated CbpA, or a derivative (e.g., fragment or fusion protein) thereof, including but not limited to rabbits, mice, rats, sheep, goats, etc. In one embodiment, the protein can be conjugated to an immunogenic carrier, e.g., bovine serum albumin (BSA) or keyhole limpet hemocyanin (KLH). Various adjuvant may be used to increase the imnmunological response, depending on the host species.

For preparation of monoclonal antibodies, or fragment, analog, or derivative thereof, any technique that provides for the production of antibody molecules by continuous cell lines in culture may be used (see, e.g., Antibodies—A Laboratory Manual, Harlow and Lane, eds., Cold Spring Harbor Laboratory Press: Cold Spring Harbor, New 1q′ York, 1988). These include but are not limited to the hybridoma technique originally developed by Kohler and Milstein (1975, Nature 256:495-497), as well as the trioma technique, the human B-cell hybridoma technique (Kozbor et al., 1983, Inmmunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, J. Bacteriol. 159-870; Neuberger et al., 1984, Nature 312:604-608; Takeda et al., 1985, Nature 314:452-454) by splicing the genes from a mouse antibody molecule specific for a protein together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves. An additional embodiment of the invention utilizes the techniques described for the construction of Fab expression libraries (Huse et al., 1989, Science 246:1275-1281) to allow rapid and easy identification of monoclonal Fab fragments with the desired specificity for the protein, or its derivatives, or analogs.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Antibodies can be labeled for detection in vitro, e.g., with labels such as enzymes, fluorophores, chromophores, radioisotopes, dyes, colloidal gold, latex particles, and chemiluminescent agents. Alternatively, the antibodies can be labeled for detection in vivo, e.g., with radioisotopes (preferably technetium or iodine); magnetic resonance shift reagents (such as gadolinium and manganese); or radio-opaque reagents.

The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. The protein can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diusocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

Vectors containing the nucleic acid-based vaccine of the invention can be introduced into the desired host by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

This invention provides a method of testing the ability of a drug, agent, or compound to modulate the activity of the protein truncate or fragment as hereinabove disclosed, which comprises: (a) culturing test cells which contain elevated levels of the protein truncate or fragment in a tumorous condition; (b) adding the drug, agent, or compound under test; and (c) measuring the change if any, in the tumorous condition of said test cells.

This invention provides a method for identifying a test composition or agent which modulates Gli 1, Gli 2, or Gli 3 or any other Gli family protein which comprises: (a) contacting a protein truncate or fragment as hereinabove disclosed with a test composition or agent under conditions permitting binding between the protein and the test composition; (b) detecting specific binding of the a test composition or agent to the proteins; and (c) determining whether the a test composition or agent inhibits the proteins, so as to identify a test composition or agent which is which modulates Gli 1, Gli 2, or Gli 3,.

This invention provides a method of identifying a test composition or agent which modulates Gli 1, Gli 2, or Gli 3 or any other Gli family, the method comprising: (a) incubating components comprising the test composition, and the proteins, wherein the incubating is carried out under conditions sufficient to permit the components to interact; and (b) measuring the effect of the test composition on the binding to the proteins.

This invention provides a method of identifying/screening a cell for protein truncates or fragments of Gli protein family comprising, introducing into the cell a protein, wherein the protein inhibits the function or activity of a protein of the Gli family; and detecting the resulting protein produced, thereby identifying/screening the cell for protein truncates or fragments of the Gli protein family. Gli family proteins include but are not limited to; Gli 1, Gli 2, Gli 3 and other homologous proteins.

This invention provides a method of inhibiting the function, or processing of Gli 1 or Gli 3, comprising introducing into a cell the protein truncates or fragments as hereinabove disclosed, or the vector comprising the nucleic acid encoding the protein truncates or fragments as hereinabove disclosed, thereby inhibiting the function of or processing of Gli 1, Gli 2, or Gli 3.

This invention provides a method for treating a subject having Polydactyly Type A (PAP-A) or Pallister-Hall Syndrome (PHS), comprising administering a therapeutically effective amount of the pharmaceutical composition comprising the protein truncates or fragments as hereinabove disclosed, or the vector comprising the nucleic acid encoding the protein truncates or fragments as hereinabove disclosed, so as to inhibit the function or processing of Gli 1 or Gli 3 expression, thereby treating the subject having Polydactyly Type A (PAP-A) or Pallister Syndrome (PHS). Other cancers include basal cell carcinoma, sarcoma, medulloblastoma, or other tumors associated or related to Gli protein activity.

This invention provides a method of obtaining a protein in purified form which comprises: (a) introducing the vector into a suitable host cell; (b) culturing the resulting host cell so as to produce the protein; (c) recovering the protein produced in step (b); and (d) purifying the protein so recovered in step (c).

This invention provides a pharmaceutical composition comprising an amount of the protein truncate or fragment and a pharmaceutically acceptable carrier or diluent.

As used herein, “pharmaceutical composition” could mean therapeutically effective amounts of protein products of the invention together with suitable diluents, preservatives, solubilizers, emulsifiers, adjuvant and/or. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Other embodiments of the compositions of the invention incorporate particulate forms protective coatings, protease inhibitors or permeation enhancers for various routes of administration, including parenteral, pulmonary, nasal and oral.

Further, as used herein “pharmaceutically acceptable carrier” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif. p. 384). Often, a primary challenge with an antigen alone, in the absence of an adjuvant, will fail to elicit a humoral or cellular immune response. Adjuvant include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvant such as BCG (bacille Calmette-Guerin) and Corynebacterium parvum. Preferably, the adjuvant is pharmaceutically acceptable.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

When administered, compounds are often cleared rapidly from mucosal surfaces or the circulation and may therefore elicit relatively short-lived pharmacological activity. Consequently, frequent administrations of relatively large doses of bioactive compounds may be required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

Dosages. The sufficient amount may include but is not limited to from about 1 μg/kg to about 1000 mg/kg. The amount may be 10 mg/kg. The pharmaceutically acceptable form of the composition includes a pharmaceutically acceptable carrier.

The preparation of therapeutic compositions which contain an active component is well understood in the art. Typically, such compositions are prepared as an aerosol of the protein delivered to the nasopharynx or as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

An active component can be formulated into the therapeutic composition as neutralized pharmaceutically acceptable salt forms. Pharmaceutically acceptable salts include the acid addition salts (formed with the free amino groups of the protein or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

According to the invention, the component or components of a therapeutic composition of the invention may be introduced parenterally, transmucosally, e.g., orally, nasally, pulmonarailly, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid).

In yet another embodiment, the therapeutic compound can be delivered in a controlled release system. For example, the protein may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Preferably, a controlled release device is introduced into a subject in proximity of the site of inappropriate immune activation or a tumor. Other controlled release systems are discussed in the review by Langer (Science 249:1527-1533 (1990)).

### EXPERIMENTAL DETAILS SECTION

### EXAMPLE 1

### Functional Dissection of Gli Proteins Identifies Multiple Products with Different Activities

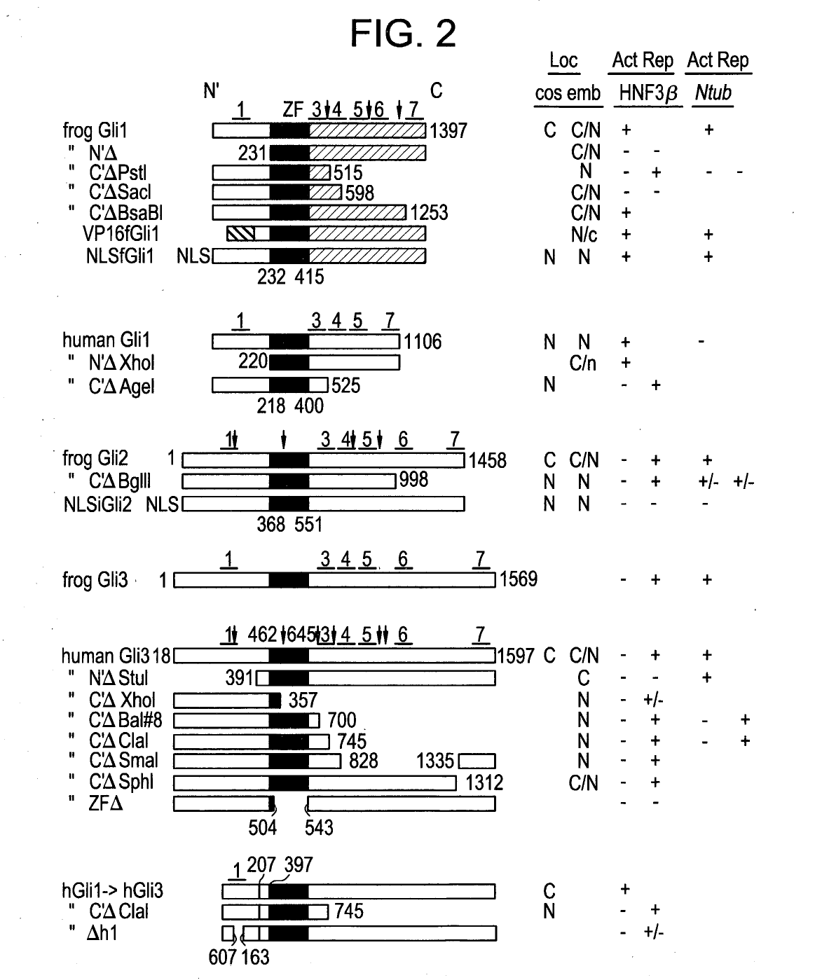

cDNA clones and synthetic RNAs: Full-length frog Gli1, frog Gli2, frog Gli3, human Gli1 and human Gli3 cDNAs in pCS2-MT (Turner and Weintraub, 1994, Genes Dev 8:1434-1447) or pcDNA-1 Amp (Invitrogen) were as described (Lee et al., 1997, Development 124: 2537-2552; Brewster et al., 1998, Nature, in press). All deletion subclones of hGli1 were in pcDNA-1 Amp with the exception of hGli1NΔXhoI which was cloned in pCS2-MT. All deletions of all other Gli genes were in pCS2-MT which provided in frame Myc-epitope tags in the N-terminus. Deletion constructs were as described in FIG. 2 with the enzyme in the name being used to generate the specified deletions. The Shh expression plasmid was as described (Ruiz i Altaba et al., 1995, Mol. Cell Neurosci 6: 106-121). A plasmid driving expression of a constitutively active type EB BMP receptor was as described (Kretzschmar et al., 1996, Genes & Dev. 11: 984-995).

Synthetic RNAs for microinjection were synthetised by linearizing the appropriate cDNA clones with NotI and transcribing with SP6 RNA polymerase for pCS2-MT clones. For pcDNA-1Amp clones this was done with NotI and T7. Transcriptions were performed with the Ambion kit at 30° C. with a trace of ³²P-UTP to measure incorporation.

Microinjection and transfection: Plasmid DNAs were microinjected into developing frog embryos at 200 pg/embryo/10 nl in water except in co-injection experiments in which 100 pg of each pDNA were injected. Microinjection of synthetic RNA was at 2 ng/embryo/10 nl unless otherwise stated. For co-injectiion experiments, each RNA was at 1 ng/embryo. Single injection controls for co-injection experiments were at 100 pg pDNA or 1 ng RNA in order to maintain the same amount of active nucleic acid. Transfection of plasmid DNA into COS cells was performed with Lipofectamine (Gibco-BRL) following standard procedures. COS-7 cells were assayed 24 hours after transfection.

Immunocytochemistry and whole-mount in situ hybridization: Transfected COS cells were immunolabeled with anti-hGli1 affinity purified polyclonal antibodies (1/100; Lee et al., 1997, Development 124: 2537-2552; Dahmane et al., 1997, Nature 389: 876-881) or with an anti-Myc monoclonal antibody (1/500-1/1000; 9E1, Santa Cruz, Inc.) followed by fluorescein- or rhodamine-coupled secondary antibodies as described (Lee et al., 1997, Development 124: 2537-2552). Nuclei were counterstained with DAPI (Sigma).

Injected embryos to be analyzed for intracellular localization of Gli proteins were processed for whole-mount immunocytochemistry using peroxidase coupled secondary antibodies and reaction with diaminobezidine as described. For viewing, however, the labeled embryos were squashed under a coverslip in benzyl alcohol-benzyl benzoate and photographed under Nomarski optics. All pictures were taken with an Axiophot microscope (Zeiss). Floor plate cells were identified by whole-mount immunoreactivity with polyclonal anti-HNF-3β antibodies as previously described (1/8000; Ruiz i Altaba et al., 1995, Mol. Cell Neurosci 6: 106-121).

Whole-mount in situ hybridization was performed according to Harland (1991, Meth. Enzymol. 36: 675-685) using maleic acid. Digoxigenin-labeled anti-sense RNA probes to N-tubulin were made by transcribing a cDNA clone with T3 after digestion with BamHI. Hybridization was revealed with anti-digozigenin alkaline-phosphatase-coupled antibodies in the presence of NBT and BCIP (BRL). After in situ hybridization, embryos were processed for whole-mount immunocytochemistry to reveal the localization of the injected Myc-tagged proteins using anti-Myc antibodies and peroxidase-coupled secondary antibodies as described above. Embryos were not cleared before viewing and pictures were taken with an M10 binocular microscope (Leica).

Immunoprecipitation and Western blotting: Immunoprecipitation and Western blotting procedures were essentially as previously described (Harlow and Lane, 1988, Antibodies. A Laboratory Manual, Cold Spring Harbor Laboratory Press, New York). In general, injected embryos were harvested at early gastrula stages (st. 10-12) and homogenized (100 μl/embryo) in RIPA buffer (150 mM NaCl, 1% NP40, 0.5% DOC, 0.1% SDS, 50 mM Tris pH 7.5) by vortexing. RIPA buffer always contained a cocktail of protease inhibitors including PMSF (Boehringer Mannheim). Insoluble material was spun out in a tabletop microcentrofuge at top speed for 10 min at 4° C. The resulting supernatant was incubated with anti-Myc antibodies (0.2 μg IgG/embryo) at 4° C. for 1 hr with constant shaking. Antigen-antibody complexes were immunoprecipitated by incubating with agarose beads coupled to protein A plus protein G (Santa Cruz) for 1 hr at 4° C. with constant shaking. The beads were washed four times with 1.5 ml RIPA buffer with constant shaking for 15 min each. Pelleted beads were boiled in sample buffer and the supernatant leaded in 7.5% SDS-PAGE gels. Electrophoresed proteins were transfered to Protran nitrocellulose membranes by standard procedures. After blocking with non-fat milk, the immunoblots were probed with anti-Myc antibodies (1/1500) and binding visualized by chemiluminescence with the ECL kit (Amersham).

Differential subcellular localization of Gli proteins in transfected COS cells versus injected frog embryos: Endogenous Gli proteins are thought to be mostly cytoplasmic (Lee et al., 1997, Development 124, 2537-2552; Dahmane et al., 1997, Nature, 389: 876-881). In transfected COS cells, tagged Gli proteins localize to the cytoplasm where they are found in apparent association with cytoskeletal components (Lee et al., 1997, Development 124, 2537-2552). However, frog gastrula embryos expressing injected Gli1 protein show both nuclear as well as cytoplasmic labeling (Lee et al., 1997, Development 124, 2537-2552). Here, the localization of Gli proteins has been reinvestigated focusing on full-length frog and human Gli1 (Kinzler et al., 1988, Nature 332: 371-374 ; Lee et al., 1997, Development 124, 2537-2552), frog Gli2 (Lee et al., 1997, Development 124, 2537-2552, Brewster et al., 1998, Nature, in press) and human Gli3 (Ruppert et al., 1990, Mol. Cell Biol. 10:5408-5415). Lacking specific high affinity antibodies to all Gli proteins, frog Gli1, Gli2 and human Gli3 proteins used in this study were tagged at their N-termini with Myc epitopes (Lee et al., 1997, Development 124, 2537-2552) in order to follow their behavior with anti-Myc antibodies. Human Glil was generally identified with affinity-purified polyclonal antibodies raised against the zinc finger region (Lee et al., 1997, Development 124, 2537-2552).

As previously shown, human Gli1 was mostly nuclear (Kinzler and Vogelstein, 1990, Mol. Cell Biol. 10: 634-642; Lee et al., 1997, Development 124, 2537-2552) whereas frog Gli1 (hereafter Gli1) was mostly cytoplasmic (data not shown) in transfected COS cells. This difference may represent an inherent difference (Dahmane et al., 1997, Nature, 389: 876-881), possibly due to the presence of a putative mutation in the glioma-derived cDNA used here (Hynes et al., 1997, Neuron 19:15-26). Both human Gli3 (hereafter Gli3) and frog Gli2 (hereafter Gli2) showed exclusive cytoplasmic localization in COS cells, with a pattern reminiscent of that of cytoskeletal components (Lee et al., 1997, Development 124, 2537-2552).

To test Gli protein localization in vivo, frog embryos were injected with plasmids driving the expression of the different Gli cDNAs and assayed injected embryos for expression at mid-gastrula stages. In frog embryos all Gli proteins showed nuclear labeling. In addition, cytoskeletal-like cytoplasmic labeling was detected with all Glis but not with human Gli1. The difference observed in the subcellular localization of Gli proteins in COS cells versus frog embryos raised the possibility that these could be efficiently modified in the embryo, giving rise to nuclear forms.

Gli proteins yield protein-specific smaller forms lacking terminal sequences: Analysis of the structure of Gli proteins produced in embryos was performed by injecting plasmid DNAs and assaying for expression of encoded Myc-tagged proteins at gastrula stages, the expression of which is driven by ubiquitously active CMV regulatory sequences. Injection of Gli1, Gli2 or Gli3 yielded full-length products that migrated in the predicted anomalous manner in denaturing-PAGE visualized by immunoprecipitation (IP) and Western blotting. For example, Gli3 is ˜1597 aa in length but migrates at 190 KD (Ruppert et al., 1990, Mol. Cell Biol. 10: 5408-5415).

In addition to these full-length products, discrete smaller bands were consistently observed in all cases. These bands are likely to be processing products as Northern analyzes detected only one or two transcripts for each gene (A. Chokas and ARA) as previously reported (Kinzler et al., 1987, Science 236: 70-73; Ruppert et al., 1990, Mol. Cell Biol. 10: 5408-5415; Hui et al., 1994, Develop.Biol. 162: 402413; Persengiev et al., 1994, Oncogene 14: 2259-2264). Only those protein products that were repeatedly detected and represented clear bands in separate independent experiments were analyzed. Because all detected proteins must contain an N-terminal tag, measuring their length would determine the approximate site of termination. However, due to the abnormal migration of Gli proteins, it was not possible to rely on standard markers for measurement. To address this issue, C-terminal deletion constructs of the Gli cDNAs were made (FIG. 2) and injected into frog embryos. Using these C-terminal deleted proteins, it was found that N-terminal sequences contributed to the abnormal migration of Gli proteins as even a small Gli3C′ΔXhoI construct lacking all C-terminal sequences and most of the zinc finger domain migrated as a ˜670 aa protein rather than the ˜560 aa protein it is. Abnormal migration, however, varied slightly in different Gli proteins as the 515 aa Gli1C′ΔPstI protein migrated slower than the 560 aa Gli3C′ΔXhoI protein. Nevertheless, the known sizes of a variety of protein-specific deletion constructs (FIG. 2) approximated the length of Gli products detected in IP/Western analyses. Gli1 consistently yielded three smaller-than-full-length bands of ˜1100, ˜760 and ˜540 aa. Gli2 consistently yielded four bands of ˜860, ˜760, ˜420 and ˜220 aa. Gli3 consistently yielded six bands of ˜1000, ˜˜760, ˜700, ˜580 and ˜340 aa. In all cases, the numbers given are +/−25 aa.

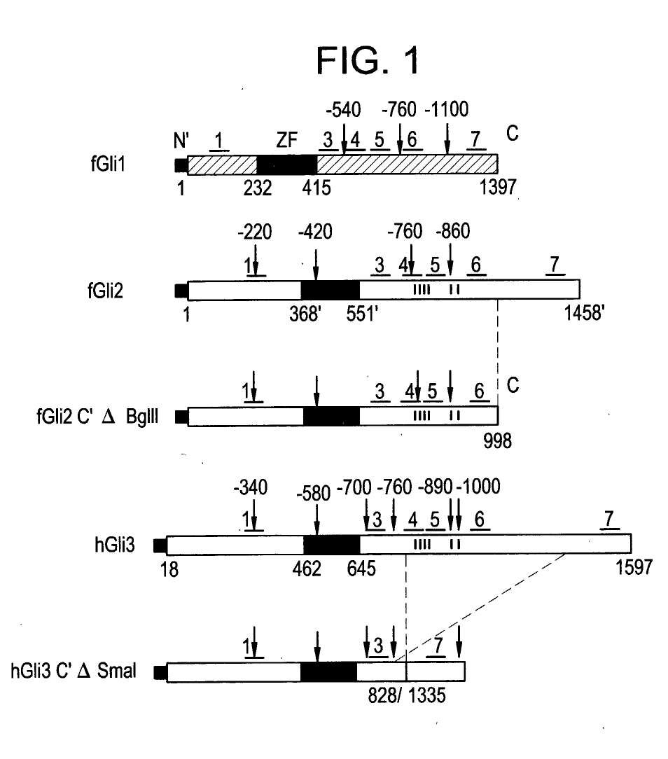

Mapping the approximate processing sites within the Gli proteins (FIG. 1) showed that even though the sizes of each product in each protein is different, there is a conservation of the overall pattern. The three Gli proteins show two or more C-terminal processing sites, one of which is near the conserved regions 5 and 6 (FIG. 2E; conserved regions are according to Ruppert et al., 1990), mapping close to putative PKA phosphorylation sites (FIG. 1). In Gli3, there are two adjacent sites that map close to two putative PKA sites in this region. In Gli1, in addition to this site which is minor, there is a major site located closer to the C-terminus. A third Gli1 site maps close to domains 3 and 4. In Gli2, there is a second C-terminal site that maps close to the putative PKA phosphorylation sites in region 4. Gli3, in contrast, shows a more complex pattern with four C-terminal sites. In addition to the aforementioned doublet in between regions 4 and 5, two prominent sites map upstream and downstream of region 3, respectively. The site mapping C-terminal to region 3 is in a similar position to that in Gli1. Gli2 and Gli3 display two additional sites. One within the DNA-binding domain, close to zinc fingers 2 or 3, and one in the N-terminus, mapping to the conserved region 1 (FIG. 1).

C-terminal deletion constructs also yielded smaller products. Gli2C′ΔBglII and Gli3C′ΔSphI constructs (FIGS. 1, 2), lacking different sequences C-terminal to region 6, showed similar patterns of putative cleavage to that of full-length proteins. In contrast, Gli3C′ΔSmaI, lacking most sequences in between regions 3 and 7 (FIGS. 1, 2), showed a variant pattern since the biggest non-full-length product resulted from processing at a novel site, close to the C-terminus (FIG. 1). To test if Gli proteins undergo rapid deletion of the very C-terminus, a double N-terminus Myc-tagged and C-terminus HA-tagged Gli3 protein was made and assayed for the production of HA-tagged smaller forms after injection. Whereas analysis with anti-Myc antibodies showed the expected pattern, anti-HA antibodies failed to recognize any proteins in IP/Westerns or in Westerns of proteins previously immunoprecipitated with anti-Myc antibodies. Thus, although the inability to detect stable HA-tagged Gli3 forms is consistent with rapid and efficient cleavage of the C-terminal end, it remains to be determined whether such a site exists.

In contrast to injected frog blastomeres, transfected COS cells did not show specific Gli products. Instead, a weak degradation ladder was observed below the full-length proteins in all cases. This observation is consistent with the cytoplasmic localization of full-length proteins in frog embryos and the cytoplasmic localization of Gli proteins in COS cells, indicating that COS cells do not process transfected Gli proteins as frog embryos do.

Differential intracellular localization of truncated Gli proteins: To test the behavior of different Gli products identified in embryos injected with tagged Gli proteins, deletion constructs were made lacking different amounts of terminal sequences, some of which mimicked the embryo-made products (FIG. 2). These truncated cDNAs were then injected into frog embryos and the encoded proteins localized inside expressing cells by whole mount immunocytochemistry.

Blastomeres expressing injected Gli1 proteins showed localization in both nucleus and cytoplasm with the exception of Gli1C′ΔPst1, a deletion construct terminating after region 3, which was exclusively nuclear. Both, a fusion of the strong transactivating domain of VP16 with an N-terminally truncated Gli1 and a Gli1 protein having an SV40 NLS sequence in its N-terminus (FIG. 2), also localized preferentially to the nucleus (Lee et al., 1997, Development 124, 2537-2552). Therefore, there is a cytoplasmic anchoring site in Gli1 that maps in or near region 4 as Gli1C′ΔPst1 is exclusively nuclear and Gli1C′ΔSacI shows nuclear as well as cytoplasmic labeling. Nuclear labeling in embryos injected with Gli1C′ΔSacI and other longer deletions could result from processing of these truncated proteins to yield smaller forms. To test if N-terminal sequences in Gli1 are involved in nuclear localization, the human Gli1 protein was used as it is nearly exclusively nuclear. Like with frog Gli1, a human Gli1 deletion lacking C-terminal sequences, hGli1C′ΔAgeI, localized to the nucleus (FIG. 2). In contrast, hGli1N′ΔXhoI (FIG. 2), an N-terminal deletion of human Gli1 bearing Myc epitopes, was found to be cytoplasmic, indicating that the amino terminus is necessary for efficient nuclear localization. However, a similar construct with frog Gli1, Gli1N′Δ also bearing Myc epitopes (FIG. 2), showed both cytoplasmic as well as nuclear labeling. Because these two constructs are nearly identical, nuclear localization could involve more than just the N-terminus.

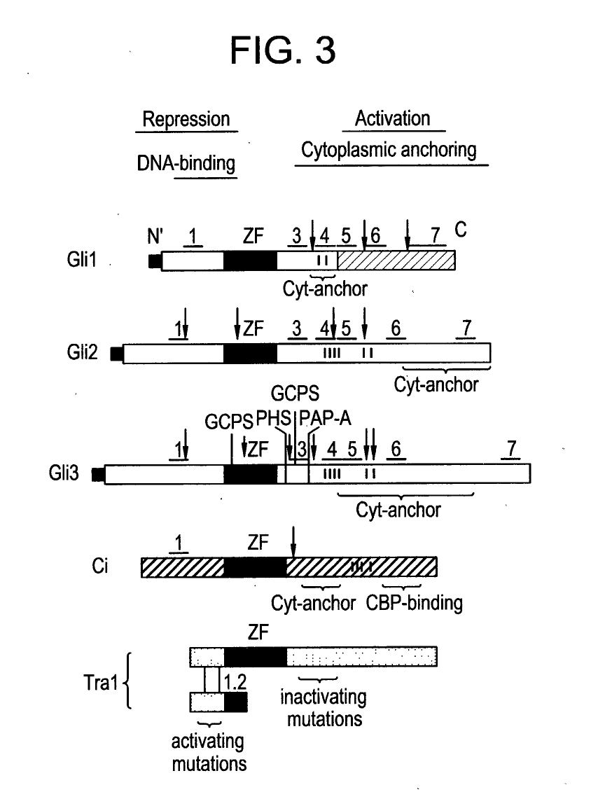

For Gli2, a C-terminal deletion lacking most sequences downstream of region 6, Gli2C′ΔBgIII (FIG. 3), was exclusively nuclear, like a nuclear-targeted construct having an SV40NLS in its N-terminus (FIG. 2). Cytoplasmic anchoring signals are thus found closer to the C-terminus in Gli2 than in Gli1. Because processing sites in Gli2 occur upstream of the point of truncation in Gli2C′ΔBglII (FIGS. 1, 2), all processed forms of Gli2 are predicted to be nuclear.

Gli3 also contains cytoplasmic anchoring sequences in the C-terminal region between regions 4 and 7 as Gli3C′ΔClaI and Gli3C′ΔSmaI were nuclear but Gli3C′ΔSphI showed both cytoplasmic and nuclear labeling (FIG. 2). Nuclear localization of Gli3 also depended on N-terminal sequences. Gli3C′ΔXhoI, lacking all sequences downstream of the second zinc finger was strictly nuclear and nuclear localization was lost when N-terminal sequences were deleted as in Gli3N′ΔStuI (FIG. 2). The dominance of the C-terminus over the N-terminus in subcellular localization became apparent in chimeric constructs (FIG. 2) in which the N-terminal part of human Gli1 was fused to the rest of Gli3. This hGli1->hGli3 protein (Ruiz i Altaba, 1998) remained cytoplasmic in COS cells but a C-terminal deletion downstream of region 3 made it become nuclear (hGli1->hGli3C′ΔClaI; FIG. 2).

Intracellular localization of Gli proteins may also be affected by interactions between Gli proteins. This is suggested by the ability of hGli1 to affect the distribution of co-transfected Gli2 or Gli3 in COS cells. Gli2 or Gli3 alone were 98% cytoplasmic and 2% nuclear (n=104; n=104), contrasting with the nuclear localization of NLSGli2. However, upon co-transfection with hGli1 , 47% of Gli2 (n=32) and 27% of Gli3 (n=82) expressing cells had nuclear label, and of these 93% and 81% co-labeled with nuclear hGli1, respectively. Gli proteins could therefore form multimers or be in a complex with other proteins, like Ci (Sisson et al., 1997, Cell 90: 235-245; Robbins et al., 1997, Cell 90: 225-234), and hGli1 may shepherd Gli2/3 to the nucleus.

Regulation of product formation: To begin to test how Gli protein processing may be regulated, Gli plasmids were targeted to the dorsal or ventral equatorial regions of the early frog embryo. These regions are known to have different active pathways such as BMP4 signaling ventrally and Shh dorsally and in midline cells. The pattern of products of Gli1, Gli2 or Gli3 was identical in dorsal and ventral regions of early or late gastrula embryos. Processing of Gli proteins was then tested after injection into the animal region along with plasmids driving the expression of either Shh (Ruiz i Altaba et al., 1995, Mol. Cell Neurosci. 6: 106-121) or a constitutively active type IB BMP receptor (Krezschmar et al., 1997, Genes & Dev. 11: 984-995; Hata et al., 1998, Genes & Dev. 12: 186-197). Enhanced Shh signaling by overproduction of the ligand did not apparently alter the pattern of products for any Gli protein. Co-injection of Gli1 with plasmids driving the expression of dominant negative PKA or constitutively active PKA subunits (Clegg et al., 1987, J. Biol. Chem. 262: 13111-13119; Orellana and McKnight, 1992, PNAS Sci. USA 89: 4726-4730) also did not consistently affect the pattern of Gli products. Enhanced BMP signaling gave a variable result. In two out of five experiments, the ˜1100 aa Gli1 product was not detectable whereas the other bands were unaffected. No consistent effects of BMP signaling were observed on Gli2 or Gli3 processing. These results suggest that BMP signaling could regulate Gli1 processing. The failure to show regulation by Shh or PKA could be due to either inability of early frog embryo cells to transduce the Shh signal efficiently or saturation of a rate-limiting step. The latter is suggested by the finding that the pattern of Gli1 was not altered when injected into ventral regions, in which BMP signaling is active.

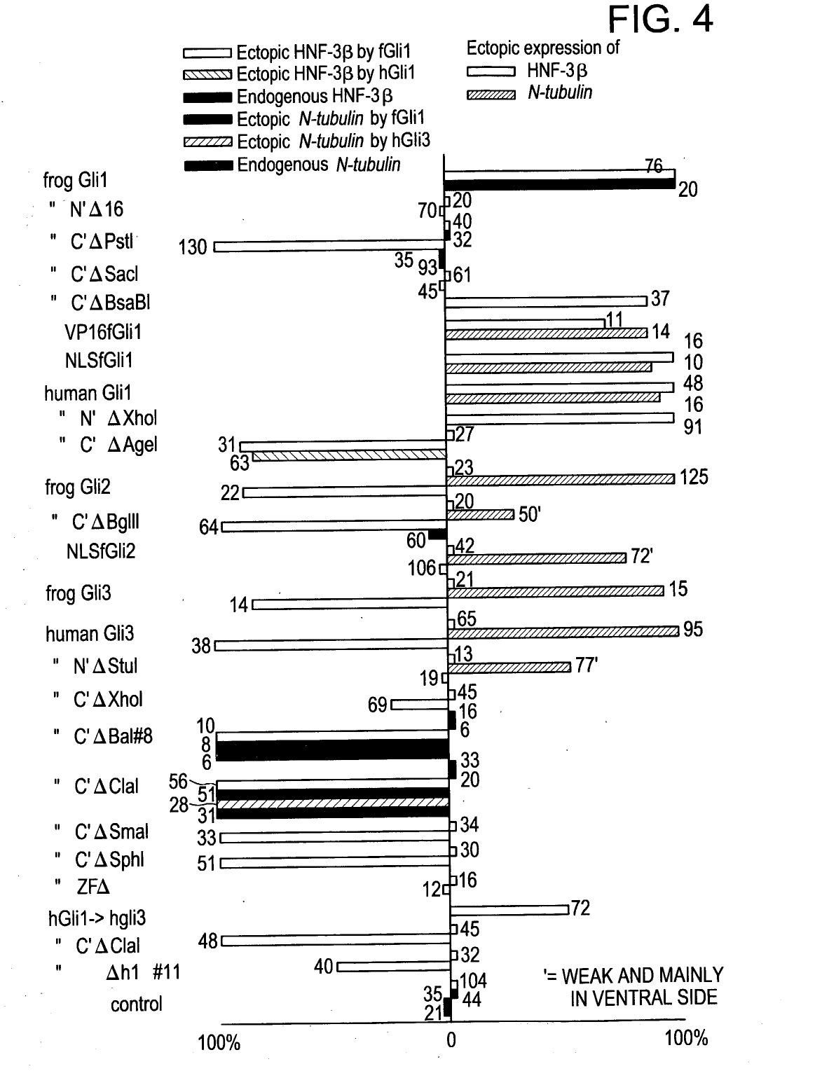

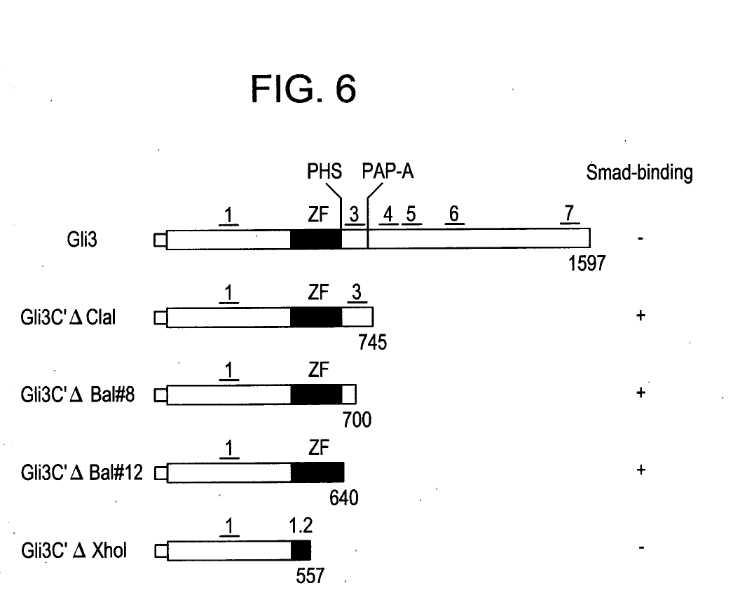

Both N- and C-terminal sequences of Gli1 are involved in regulating floor-plate-inducing activity: To test the functional significance of the different forms of Gli1, the ectopic floor plate induction assay was used (Ruiz i Altaba et al., 1995, Mol. Cell Neurosci. 6: 106-121; Lee et al., 1997, Development 124: 2537-2552; Ruiz i Altaba, 1998, Development 125: 2203-2212), injecting synthetic RNAs and recognizing floor plate and immediately adjacent cells by the expression of HNF-3β protein. Ectopic expression of Gli1 leads to the ectopic differentiation of floor plate cells expressing HNF-3β (Table1; Lee et al., 1997, Development 124: 2537-2552), not observed in control sibling embryos (Table 1). Injection of VP16Gli1 or NLSGli1 also resulted in ectopic HNF-3β expression (FIG. 2; Table 1; Lee et al., 1997, Development 124: 2537-2552), as did a C-terminal deletion lacking region 7 (Gli1C′ΔBsaBI, FIG. 2; FIG. 4). However, floor plate-inducing activity was lost in proteins lacking C-terminal sequences downstream of region 4 (Gli1C′ΔSacI; FIGS. 2 and 4). An N-terminal truncation (Gli1N′) also lacked floor-plate-inducing activity (FIGS. 2 and 4). In human Gli1, C-terminal sequences are also required for floor-plate-inducing activity (hGli1 C′ΔAgeI; FIGS. 3 and 6) but a similar N-terminal deletion to that in frog Gli1 maintained activity (hGli1N′XhoI, FIG. 2; Table 1). Neither full-length (Lee et al., 1997, Development 124: 2537-2552; Ruiz i Altaba, 1998, Development 125: 2203-2212) nor any deletion proteins or Gli2 or Gli3 had floor-plate-inducing activity (FIGS. 2 and 4). However, the N-terminal domain of human Gli1 was able to confer activity to the hGli1->hGli3 chimera (FIG. 2; Table 1; Ruiz i Altaba, 1998, Development 125: 2203-2212), and this was dependent on C-terminal sequences on Gli3 as a C-terminal deletion (hGli1->hGli3C′ΔClaI) lacked activity (FIG. 2; Table 1). Within the N-terminus, activity depended on the presence of region 1 as its deletion from hGli1->hGli3 abolished inducing activity (FIG. 2; Table 1). Absence of region 1, in addition, turned an activator into a partial repressor (Table 1). These results raise the possibility that the Gli regulation involves intramolecular interactions.

C-terminal deletions of Gli1 turn activators into dominant repressors: The lack of floor-plate-inducing activity in C-terminal deletions of Gli1 and of hGli1->hGli3, missing sequences downstream of region 4 raised the possibility that they could, like in the case of Ci, act as repressors. To test this idea, plasmids driving the expression of full-length Gli1 were co-injected with plasmids driving the various truncated forms, and the injected tadpoles assayed for the repression of ectopic HNF-3β expression, normally induced by the injected full-length Gli1. Co-injection of Gli1C′ΔPst1, but not of Gli1C′ΔSacI, inhibited ectopic HNF-3β expression by co-injected full-length Gli1 (FIG. 2; Table 1). This result indicates that Gli1 proteins lacking C′ sequences downstream of region 3 can act as dominant negative. Gli1C′ΔPstI was, however, unable to inhibit endogenous floor plate differentiation when targeted to the midline (Table 1). Interestingly, addition of region 4, as in Gli1C′ΔSacI, inhibited repressor function without introducing positive floor-plate-inducing activity (FIG. 2; Table 1). Analysis of human glioma Gli1 protein function in co-injection assays with frog Gli1 showed the same effect. Deletion of C-terminal sequences downstream of region 3, yielding hGli1C′ΔAgeI, turned an activator into a dominant repressor (FIG. 2; Table 1). The N-terminus of Gli1 appears to be required for repressor activity as co-injection of the N-terminal truncated construct Gli1N′Δ failed to inhibit ectopic floor plate induction by co-injected full-length Gli1 (FIG. 2; Table 1).

Repression of Gli1 function by Gli2 or Gli3 is encoded in the N-terminus: Gli2 and Gli3 are unable to induce HNF-3β expression and instead suppress endogenous and Gli1-induced ectopic floor plate cell differentiation (Ruiz i Altaba, 1998, Development 125: 2203-2212). Full-length Gli2 inhibited floor plate induction by co-injected full-length Gli1 (FIG. 2; Table 1). Co-injection of the C-terminal deletion Gli2C′ΔBgIII also inhibited Gli1 function (FIG. 2; Table 1). However, a nuclear targeted NLSGli2 protein failed to inhibit Gli1 function (FIG. 2; Table 1), showing that the full-length protein is not a repressor and suggesting that it needs to be processed in the cytoplasm in order to act as such.

The idea that repression of Gli1 function by Gli2 and Gli3 is encoded in the N-terminus is further supported by the results with Gli3 deletions. All C-terminal deletions repressed floor-plate-inducing function of Gli1 in co-injection assays and an N-terminal deletion of Gli3, Gli3N′ΔStuI, failed to repress Gli1 function (FIG. 2, Table 1). Failure of a Gli3 protein lacking the zinc finger domain (Gli3ZFΔ to inhibit Gli1 function (FIG. 2, Table 1; Ruiz i Altaba, 1998, Development 125: 2203-2212) suggested that repressor function requires DNA-binding. Interestingly, however, a Gli3 protein consisting of the N-terminus and only the first two zinc fingers (Gli3C′ΔXhoI) was able to partially inhibit floor plate induction by co-injected Gli1 (FIG. 2; Table 1). The Gli3C′ΔXhoI protein is unlikely to bind DNA as it lacks the last three DNA-binding fingers (Pavletich and Pabo, 1993, Science 261: 1701-1707; Zarkower and Hodgkin, 1993, Nucleic Acids Research 21: 3691-3698), raising the possibility that there could be two distinct ways in which the N-terminus may act as a repressor, one of them being DNA-binding-independent.

C-terminal sequences are required for neuronal induction: In contrast to the restricted ability of Gli1 to induce floor plate development, all three Gli genes can induce neuronal differentiation (Lee et al., 1997, Development 124: 2537-2552; Brewster et al., 1998, Nature, in press; Ruiz i Altaba, 1998, Development 125: 2203-2212). Moreover, Gli function is required for endogenous primary neuronal development (Brewster et al., 1998, Nature, in press ). The differentiation of primary neurons, as identified by expression of N-tubulin (Oschwald et al., 1991, Int. J. Dev. Biol. 35: 399-405), was therefore used as an assay to study structure/function relationships in relation to positive inducing actions of Gli2 and Gli3.

Expression of either one of the three full-length Gli proteins from injected RNAs resulted in neuronal differentiation, which was most pronounced with Gli2 (FIG. 2, Table 1). Expression of a nuclear-targeted NLSGli2 also resulted in ectopic neuronal differentiation (FIG. 2, Table 1) although this was considerably weaker than with Gli2, perhaps indicating that function as a potent activator requires cytoplasmic alterations. Expression of Gli2C′ΔBgIII also resulted in weak neuronal induction (FIG. 2; Table 1) indicating that robust positive action of Gli2 in neuronal induction involves a protein containing C-terminal sequences missing in Gli2C′ΔBgIII.

Analysis of Gli3 showed also that C-terminal sequences are required for ectopic neuronal induction as Gli3C′ΔClaI and Gli3C′ΔBal#8 failed to induce neuronal differentiation (FIG. 2; Table 1). In addition, N-terminal sequences, which are required for repression of floor plate induction by Gli 1, are not required for neuronal induction as an N-terminal deletion of Gli3, Gli3N′ΔStuI, induced neuronal differentiation, albeit slightly less efficiently than the full-length protein (FIG. 2; Table 1). As with Gli2 or Gli3, deletion of C-terminal sequences of Gli1, as in Gli1 C′ΔPstI, also resulted in the loss of neuronal-inducing activity (FIG. 2; Table 1). Induction of neuronal differentiation by all three Gli genes, thus appears to involve nuclear proteins containing C-terminal sequences, some of which could lack the N-terminus.

C-terminal deletions uncover dominant repressors of neuronal differentiation: The loss of activity in Gli proteins lacking C-terminal sequences raised the possibility that these could be acting as dominant repressors of neuronal differentiation, much like in the case of floor plate induction. To assay repressor activity, co-injection assays with C-terminal deleted constructs plus full-length Gli2 or Gli3 were performed. Co-injected Gli2C′ΔBgII had a minor negative effect on the activity of Gli2 (FIG. 2; Table 1), indicating that loss of robust positive inducing activity is not correlated with the gain of robust dominant-negative activity. Processed forms of Gli2 may be strong repressors as Gli2 requires cytoplasmic modification to inhibit Gli1 function. These results also suggest that repression of ectopic floor plate and neuronal differentiation may be quantitatively or qualitatively distinct functions.

To further test for the existence of dominant-negative repressors of neuronal differentiation, proteins lacking most of the C-terminus in Gli1 and Gli3 as these are repressors of ectopic floor plate induction and approximate processed forms were focused on (FIG. 2). In co-injection assays, Gli1C′ΔPstI lacked dominant negative activity in neuronal induction (FIG. 2; Table 1). In contrast, a similar Gli3 C-terminal truncation, Gli3C′ΔClaI, as well as a protein lacking almost all C-terminal sequences, Gli3C′ΔBal#8, acted as dominant repressors of neuronal induction in co-injection assays with full-length Gli3 or Gli2 (FIG. 2; Table 1).

The activities of Gli1C′ΔPstI and Gli3C′ΔClaI were further tested by single injections and assaying for endogenous neuronal differentiation. Expression of Gli1C′ΔPstI did not interfere with endogenous neuronal differentiation (Table 1). Expression of Gli3C′ΔClaI, however, caused a complete repression of primary neuronal differentiation in cells inheriting the Myc-tagged injected protein (Table 1). Gli3, but not Gli1, can thus produce a strong dominant repressor of neuronal differentiation.

As shown in FIG. 4, injected embryos analyzed in different assays as depicted on the top were scored and the results are presented in horizontal histograms in percentages. The total number of embryos assayed in each case is given in brackets next to the histogram. Activation assays are shown to the right and repression assays are shown to the left. See text and FIG. 2 for details on proteins structure.

### Discussion

Functional conservation and divergence of vertebrate Gli genes and their products: Here it is shown that tagged Gli genes injected into developing frog embryos yield full-length and smaller products with divergent activities. This suggests that endogenous Gli proteins are processed to yield proteins with variant functions although how the pattern of processed tagged Gli proteins compares to the endogenous one in different regions or tissues remains to be determined. Assays in frog embryos with truncated proteins have assigned predicted functions to different C-terminally deleted processed forms (FIGS. 2, 3). Full-length Gli1 and a smaller product lacking C-terminal sequences just upstream of region 7 are predicted to be positive transactivators, even though they are mostly cytoplasmic. In contrast, the smallest form, terminating after region 3, is a weak nuclear repressor. For Gli2, proteins containing the entire C-terminus may be the only positively acting forms in neuronal induction. Those lacking C-terminal sequences downstream of region 4 and 5 lack neuronal-inducing activity and could represent nuclear repressor forms as Gli2 does not appear to be processed to yield a form terminating after region 3, equivalent to those of Gli 1 and Gli3. Consistent with this difference, the cytoplasmic anchoring domain in Gli2 is located downstream of region 6 whereas in Gli1 it is found in or around region 4. For Gli3, positively activating forms also appear to contain most of the C-terminus and forms lacking C-terminal sequences downstream of the zinc finger domain with or without region 3 are dominant nuclear repressors.

Stable C-terminally deleted forms of Gli2 or Gli3 lacking either all or the last three zinc fingers are unlikely to bind DNA. Indeed, a short form of the C.elegans Gli family member Tral having only the first two fingers fails to bind DNA and has been thought to enhance the function of activating forms by sequestering putative negative regulators (FIG. 3; Zarkower and Hodgkin, 1993, Nucleic Acids Research 21: 3691-3698). A similar form of Gli3 (Gli3C′ΔXhol), however, acts as a mild repressor, suggesting that repressors could also act in a DNA-binding-independent manner. Such an effect could be consistent with the partial co-localization of co-transfected Gli proteins in COS cells which suggests that Gli proteins may form multimers. Perhaps N-terminal pieces inhibit positive Gli function by forming inactive complexes with activating forms.

Endogenous Gli2 and Gli3 proteins lacking most or all of the N-terminus may be stable and constitute activating, mostly cytoplasmic forms. This is suggested by the finding that Gli3 retains neuronal inducing activity in the absence of the N-terminus, and that there are processing sites within region 1 and near fingers 2/3. In Gli1, however, the N-terminus is required for floor plate-inducing activity and there is no processing within this region.

The present analyzes have also uncovered functions for some of the six conserved domains in all Gli proteins in addition to the zinc finger DNA-binding domain (which is included in region 2; FIGS. 2, 3). In the N-terminus of Gli1, region 1 is required for full floor plate-inducing activity. The function of region 3 is unknown although in Gli3 it plays a critical role in human disease (see below). In Gli1 region 4 and/or adjacent sequences encode a cytoplasmic anchoring domain that in its absence allows the function of a nuclear localization mechanism dependent on N-terminal sequences. In Gli2, however, cytoplasmic anchoring sequences are closer to the C-terminus. The function of regions 5 and 6 is unclear. Region 7, which may form an amphipathic helix and can act as a transactivating domain (Yoon et al., 1998, J. Biol. Chem. 273: 3496-3501), is dispensable in Gli1 for floor-plate-inducing activity.

Repressor function in the N-terminus is masked in the activating forms, being functional only when C-terminal sequences are absent. This, and the ability of the N-terminus of human Gli1 to confer floor-plate-inducing activity to a Gli1->Gli3 chimera, which nevertheless depends on Gli3 C-terminal sequences for its activity, raise the possibility that regulation of Gli protein function involves cross-talk between the N- and C-termini.

Parallels between Gli and Ci proteins: The processing of tagged-Gli proteins in frog embryos suggests a parallel with that of endogenous Ci in fly embryos. Ci is cleaved at a site downstream of the zinc finger domain to yield a nuclear repressor form (Aza-Blanc et al., 1997, Cell 89: 1043-1053), as the activation and cytoplasmic anchoring domains of Ci are found in the C-terminus (Alexandre et al., 1996, Genes & Dev. 10: 2003-2013; Hepker et al., 1996, Development 124: 549-558; Aza-Blanc et al., 1997, Cell 89: 1043-1053). This form is equivalent to the smallest repressor forms of Gli1 and Gli3 with the exception that region 3 is not recognizable in Ci. The activating form of Ci, the full-length protein or a modified form, binds the co-activator CBP and together transactivate Hedgehog/Ci target genes (Akimaru et al., 1997, Nature 386, 735-738). The CBP-binding domain of Ci is found in the C-terminus, and an equivalent position in Glis would be between regions 6 and 7. Perhaps this is the reason why C-terminal sequences are required in Gli proteins for positive inducing functions. Moreover, CBP binding to Glis could also inhibit any possible repressor function of the N-terminus. The paradox that positive transactivating Gli/Ci forms are abundant in the cytoplasm remains unresolved. It is possible that very small amounts of protein translocate to nuclei and are sufficient for function (Kelsey-Motzney and Holmgren, 1995, MOD 52: 137-150), or that nuclear activators are masked by their association with co-factors such as CBP. The existence of multiple forms for each Gli protein contrasts with the single processed form of Ci (Aza-Blanc et al., 1997, Cell 89: 1043-1053) and suggests that the vertebrate Gli family encodes a highly evolved functional repertoire.

The processing of Gli proteins observed in injected frog embryos raises the possibility that regulation of endogenous processing is critical for function. Ci processing is inhibited by Hedgehog signaling (Aza-Blanc et al., 1997, Cell 89: 1043-1053), thus allowing activation of Hedgehog target genes. It is not clear whether Gli processing is regulated by Shh signaling. However, processing sites for tagged Gli2/Gli3 map close to PKA sites, suggesting that processing could be modulated by PKA activity, as in the case of Ci (Chen et al., 1998, PNAS Sci. USA 95: 2349-2354). This would be consistent with the negative effect of PKA on Shh signaling (Fan et al., 1995, Cell 81: 457465; Hammerschmidt et al., 1996, Gene & Dev. 10: 647-658; Epstein et al., 1996, Development 122: 2885-2894; Concordet et al., 1996, Development 122: 2835-2846; Epstein et al., 1996, Development 122: 2885-2894; Ungar et al., 1996, Dev. Biol. 178: 186-191). Moreover, it is possible that some negative effects of PKA on Shh function could be mediated indirectly on Gli 1 by promoting the formation of dominant repressor forms of co-expressed Gli3. Inhibition of the production of an activating and thus Shh-mediating form of Gli1 by BMP signaling suggests an integration of antagonistic Shh and BMP pathways (Liem et al., 1995, Cell 82: 969-979) at the level of Gli protein function.

Dominant-negative products and endogenous roles of Gli proteins in floor plate and neuronal development: A Gli protein lacking C-terminal sequences downstream of region 3, Gli1C′ΔPstI, approximates an in vivo processed form and acts as a repressor of ectopic floor plate induction by co-injected full-length Gli1. This truncated form, however, is unable to abolish endogenous HNF-3β⁺ floor plate differentiation when targeted to the midline, where Gli1 is the only Gli gene expressed endogenously. This result contrasts with that obtained when full-length Gli2 or Gli3 are targeted to the midline, resulting in the loss of floor plate differentiation (Ruiz i Altaba, 1998, Development 125: 2203-2212). It is possible that overexpression of a Gli1 repressor form may not overcome the function of the endogenous activating Gli1 protein in prospective floor plate cells, perhaps due to possible modifications in these cells or the action of putative Gli1 co-factors. It is also possible that Gli1C′ΔPstI is an inherently weak repressor of endogenous functions as it also fails to inhibit primary neuronal differentiation, a process known to depend on Gli function (Brewster et al., 1998, Nature, in press). In mice, a requirement of Gli function in floor plate development is supported by the requirement of Gli-binding sites to activate HNF-3β expression in transgenic mice (Sasaki et al., 1997, Development 124: 1313-1322) although Gli1 mutant mice appear normal (Mo et al., 1998, Development 124: 113-123). Another possibility is that Shh signaling may trigger two parallel pathways involved in floor plate induction, with one being Gli1-independent and sufficient for normal development. In this case, Gli2/3 would have to do more than just repressing Gli1 function when targeted to the midline, perhaps recruiting cells to alternate non-midline fates. Existence of a non-Gli 1 pathway involved in floor plate differentiation could be supported by the action of parallel non-Ci/Gli pathways in the regulation of two Hedgehog-target genes (Krishnan et al., 1997, Science 278: 1947-1949; Lessing and Nusse, 1998, Development 125: 1469-1476).

Within the early frog neural plate, the combined expression of Gli genes encompasses the entire neural plate but neurons differentiate only in three bilateral stripes. Gli function is required for primary neurogenesis (Brewster et al., 1998, Nature, in press), a finding also supported by the ability of a truncated dominant repressive Gli3 protein to inhibit endogenous neuronal differentiation. It is possible that there is a regional production/action of repressor Gli proteins in the inter-stripe areas. In secondary neurogenesis, Gli2 can induce the differentiation of ectopic motor neurons within the neural tube (Ruiz i Altaba, 1998, Development 125: 2203-2212). This contrasts with the apparent normal development of motor neurons in Gli2 mutant mice (Matise et al., 1998, Development, in press), in which the Gli2 coding sequence is disrupted downstream of finger 2, like in the Gli1 mutants (Mo et al., 1997, Development 124: 113-123). The present results, however, indicate that if protein were to be made from the disrupted Gli2 locus, it could have dominant-negative effects on other Gli proteins. Thus, the possibility remains that the loss of floor plate in Gli2 mutant mice (Matise et al., 1998, Development, in press) results from an inhibition of Gli1 function by a putative truncated Gli2 protein. For motor neuron induction, however, there could be Gli and non-Gli parallel pathways triggered by Shh.

Dominant human mutations in Gli3 produce dominant repressors: Loss of function of Gli3 in mice and humans yields an embryonic lethal phenotype (Greig's cephalopolysyndactyly syndrome in humans, GCPS) that includes limb, facial, skeletal and neural tube malformations (Vortkampt et al., 1991, Nature 352: 539-540; Schimmang et al., 1992, Development 116: 799-804; Hui and Joyner, 1993, Nature Genetics 3: 241-246; Franz et al., 1994, Acta Anat 150: 38-44; Byscher at al., 1997, Mech. Dev. 62: 175-182; Mo et al., 1997, Development 124: 113-123; Masuya et al., 1997, Dev. Biol. 182: 42-51). In addition, two dominant mutations in human Gli3 have been recently characterized that result from truncations of the protein within the C-terminus (FIG. 3; Kang et al., 1997, Nature Genet. 15, 266-268; Radhakrishna et al., 1997, Nature Genetics 17: 269-271). One is predicted to produce a protein terminating before region 3 and produces Pallister-Hall syndrome (PHS), a pleiotropic lethal syndrome distinct from, but overlapping with, GCPS (Kang et al., 1997, Nature Genet. 15: 266-268). The second one is predicted to yield a protein terminating after region 3 and produces polydactyly type A (PAP-A) with a sixth well-formed post-axial toe and finger (Radhakrishna et al., 1997, Nature Genetics 17: 269-271).

The results presented here show that the sites of truncation of Gli3 in PHS and PAP-A map close to processing sites for injected proteins (FIG. 3) suggesting that PH and PAP-A result from the deregulated production of near-normal dominant repressor forms. Moreover, C-terminal deletions approximating the PHS and PAP-A mutations are dominant nuclear repressors of all Gli functions tested, indicating that these diseases involve repression of all activating Gli function in regions that express the Gli3 gene. However, the striking difference of the human phenotypes in PAP-A and PHS, as well as its requirement for normal function (Wild et al., 1997, Hum. Mol. Genet. 6: 1979-1984), implicates region 3 as a key element in Gli3 function. Addition of region 3 could alter the function of the PHS repressor. Alternatively, the PAP-A form could be processed to yield a PHS-like repressor only in limbs. The predicted existence of multiple endogenous processed repressor and activating forms for Gli3 suggests an exquisite control of repression function the deregulation of which leads to disease.

### EXAMPLE 2

### Gli Proteins Affect Pattern Formation Induced by TGFβ Pathways and Associate with Smads

Plasmids: Frog Gli 1 and Gli2 and human Gli3 cDNA constructs were in pCS2-MT, bearing Myc tags in the N-terminus have been described^{1,11}. Smad plasmids and activated receptor constructs were described previously: Flag-Smad1 (Liu et al. 1996, Nature 381: 620-623), Flag-Smad2, Flag-Smad3 and Flag-Smad4 (Chen et al. 1996, Nature 383: 691-696), BMPRIA (Q233D; Hoodless et al., 1996, Cell 85: 489-500), and TβRI (T204D; Wieser et al. 1995, EMBO J. 14: 2199-2208).

Embryos and microinjection: Xenopus laevis embryos were reared by standard techniques. Microinjection of synthetic RNAs was at 2 ng/10 nl/embryo. Full-length sense Gli RNAs for microinjection were made with the Ambion kit by digesting the pCS2-MT Gli clones with NotI and transcribing with SP6 RNA polymerase.

Whole-mount immunocytochemistry, in situ hybridization and histology: Embryos were processed by in situ hybridization using maleic acid. Anti-sense digoxigenin-labeled RNA probes were made as follows. A Gli3 cDNA was cut with NotI transcribed with T3 (Lee et al. 1997, Development 124: 2537-2552). A Pintallavis cDNA was digested with HindIII and transcribed with T7 (Marine et al. 1997, Mech. Dev. 63: 211-225). A Shh cDNA was digested with NotI and transcribed with T3 (Ruiz I Altaba et al, 1995 Mol. Cell. Neurosci. 6: 106-121). An Msx1AEHD cDNA was digested with EcoRI and transcribed with T3 (Suzuki et al. 1997, Development 124: 3037-3044). Antibobies used in whole-mount labeling were polyclonal anti-HNF-3β antibodies used at 1/8000 (Ruiz I Altaba et al, 1995 Mol. Cell. Neurosci. 6: 106-121) and the pan-neural monoclonal Xenl used at ⅕ (Ruiz I Altaba et al., 1992, Development 115: 67-80). Labeled embryos were either sliced with a surgical scapel or embedded in paraplast and sections (12 μm) cut in a microtome.

Immunoprecipitation and immunoblotting: The assay was performed as previously described ( Liu et al, 1997, Genes Dev. 11:3157-3167). RIB/L17 or COS1 cells were cotransfected with Myc-tagged full-length or deletion derivatives of Gli3 or Gli1 along with a Flag-tagged Smad construct by the DEAE-dextran method. BMP or TGFP stimulation was by cotransfection of an activated BMP or TGFβ receptor and treatment with BMP (2 nM) or TGFβ (500 pM) for 1 hour. Cells were lysed in TNE buffer (10 mM Tris (pH7.8), 150 mM NaCl, 1 mM EDTA, 1% NP40) in the presence of protease inhibitors. Immunoprecipitation was performed by incubation with the M2 anti-Flag antibody (Eastman Kodak) for 1 hour. Immunoprecipitates were separated in an 8% SDS-PAGE and transferred to a PVDF membrane. Inmmunoblotting was carried out by using the 9E10 anti-Myc antibody (ascites), followed by incubation with HRP-conjugated goat anti-mouse antibodies and detected by chemiluminescence (Amersham).

### Results

Elucidating how Hedgehog and TGFβ family signals are integrated is critical to understand the patterning of many tissues in the vertebrate embryo. Integration could occur at the level of Gli function since i) Gli1 and Gli2 respond to and mediate Shh signaling whereas Gli3 is an antagonist (Lee et al., 1997, Development 124: 2537-2552; Sasaki et al, 1997, Development 124: 1313-1322; Ruiz I Altaba, 1998, Development 125: 2203-2212). ii) Gli2 and Gli3 are expressed adjacent to many sites of BMP signaling, including the early ventral/posterior mesoderm (Lee et al., 1997, Development 124: 2537-2552; Sasaki et al, 1997, Development 124: 1313-1322; Marine et al, 1997, Mech. Dev. 63: 211-225), a cell group known to require BMP signaling for its development (Graff et al , 1994, Cell 79: 169-179; Hemmati-Brivanlou et al, 1995, Dev. Genet. 17: 78-89; Harland et al., 1997, Ann. Rev. Cell. Dev. Biol. 13: 611-667). iii) Genetic analyses (Dunn et al, 1997, Dev. Biol. 188: 235-247) have suggested an interaction between Gli3 and BMP4 . Here, the hypotheses that Gli2 and Gli3 affect TGFβ-induced mesodermal patterning and that Glis interact with Smads, the latter being the intracellular transducers of TGFβ family signals was tested (Heldin et al., 1997, Nature 390: 465-471; Massagu et al, 1998, Ann. Rev. Biochem. 67: 753-791). It is shown that deregulated expression of full-length Gli3 and to a lesser extent Gli2, but neither Gli1 nor repressor derivatives (Ruiz I Altaba 1992, Development 115: 67-80), induces partial posterior secondary axes potentiating intermediate mesoderm, and inhibit dorsal Spemannos organizer development. Moreover, Gli3, but not Gli1, interacts with Smads but such interactions occur only with C-terminal truncated dominant repressor forms (Ruiz I Altaba 1992, Development 115: 67-80). TGFβ family signaling induces dissociation of Gli-Smad complexes, possibly coordinating the separate but concerted action of both proteins. The findings suggest an integration of Hedgehog and TGFβ family pathways at the level of Gli-Smad function and point to novel molecular mechanisms underlying two Gli-associated human diseases.

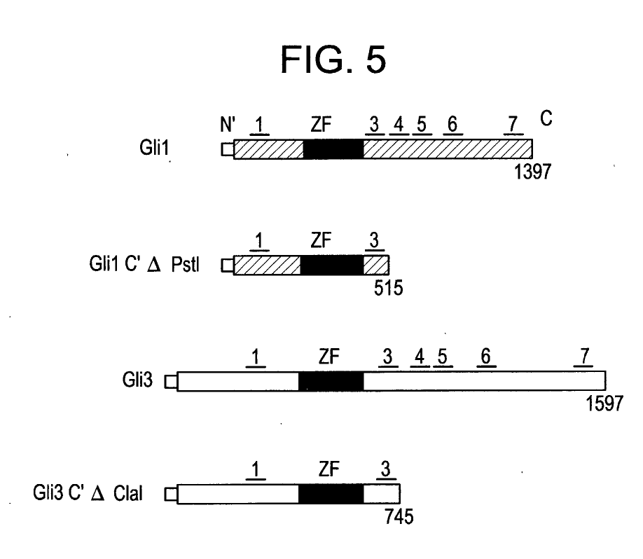

Embryos injected with Gli2 or Gli3, but not Gli1 or uninjected controls, showed the development of axial protrusions. These partial posterior axes derived from either the flank or the dorsal side and contained mesodermal and ectodermal tissues including some Xen1⁺ neural tissue (Ruiz I Altaba et al., 1995, Mol. Cell. Neurosci. 6: 106-121) but not HNF-3β⁺ floor plate cells (Ruiz I Altaba et al. 1995, Mol. Cell. Neurosci. 6: 106-121). In contrast, Gli1-injected embryos showed massive ectopic neural differentiation but no duplicated axes. The secondary axes did not contain notochord but had instead large amounts of mesenchyme connected to the lateral plate and/or somites. Secondary axes were not detected in embryos injected with Gli3C′ΔClaI, a C-terminal truncated repressor form of Gli3 (FIG. 5; n=15). Taken together, these results resemble those obtained after inhibition of ventral BMP signaling (Graff et al, 1994, Cell 79: 169-179), raising the possibility that full-length Gli3 and possibly Gli2 modify ventral BMP action.

Effects of Gli proteins on mesodermal patterning induced by other TGFβ family members were tested by assaying for their effects on dorsal differentiation, since this is thought to depend on Activins and Nodals as well as inhibition of BMP signaling (Harland et al., 1997, Ann. Rev. Cell. Dev. Biol. 13: 611-667). Dorsal expression of Gli1 did not alter Pintallavis expression normally found in Spemannos organizer of early gastrula embryos and induced by Activin/Nodal signaling (Ruiz I Altaba et al, 1992, Development 116: 81-93). However, expression of either Gli2 or Gli3 inhibited Pintallavis expression in a cell-autonomous manner. The ventral mesodermal marker Msx1 (Suzuki et al., 1997, Development 124: 3037-3044) was not ectopically expressed in the dorsal side of Gli-injected embryos at gastrula or neurula stages (n=12 each). Gli-injected embryos were also tested for the expression of Shh mRNA (Ruiz I Altaba et al, 1995, Mol. Cell. Neurosci. 6: 106-121; Ekker et al., 1995, Development 121: 2337-2347) in the notochord, a derivative of Spemannos organizer. Dorsal expression of Gli2 or Gli3, but not Gli1, inhibited Shh expression which was correlated also with loss of dorsal morphological differentiation. Myc-tagged Gli1- but not Gli2- or Gli3-expressing cells were detected in midline Shh-expressing areas. These results are consistent with a modification of both ventral and dorsal TGFβ family signaling by Gli proteins.

To test if the repressive effect of Gli3 on organizer differentiation can be mediated by its repressor products, embryos were injected with the Gli3C′ΔClaI repressor. Dorsal, but not ventral, expression of this Gli3 repressor protein inhibited Pintallavis and Shh expression. In these embryos, the ventral marker Msx1 was not ectopically expressed in dorsal regions showing that Gli3C′ΔClaI mimics the action of the full-length protein. To test if inhibition of dorsal differentiation required the DNA-binding domain, a Gli3 protein lacking all C-terminal sequences and the last three zinc fingers, Gli3C′ΔXhoI, was injected and found to be unable to repress Pintallavis or Shh expression, although some embryos showed a minor decrease in the level of marker expression. Gli3, possibly through its in vivo processed smaller DNA-binding repressor derivatives, thus appears to act as an antagonist of dorsal mesodermal development.

Possible interactions between Gli and Smad proteins were investigated using an established in vitro system in which association of co-transfected proteins is detected by immunoprecipitation and immunobloting (Liu et al., 1997, Genes Dev. 11: 3157-3167). Expression of full-length Gli constructs in COS1 or R1B/L17 cells resulted in the production of full-length proteins and a minor ladder of degradation products. Transfection of full-length Gli cDNAs identified Smad-interacting proteins of variable length which were always smaller than full-length Gli products in cells transfected with full-length Gli2 or Gli3 but not Gli1 constructs Degradation products of Gli2 and Gli3 lacking C-terminal sequences, but not the full-length proteins, could therefore bind Smads, even though the size of these products was variable and were present at very low levels. The exclusive interaction of Smads with smaller than full-length Gli proteins suggest a complex structural aspect of Gli function.

To clarify the interactions of smaller than full-length Gli products with Smads, two C-terminal truncated forms of Gli3 and Gli1 that approximate processed products in frog blastomeres and act as dominant repressors were used (FIG. 2). Expression of these proteins showed that they are stable in R1B/L17 cells and Gli3C′ΔClaI, but not Gli1C′ΔPstI, specifically associated with Smads. The interaction between Gli3 repressor protein and Smads is expected to be physiologically relevant, as the proportion of Gli3C′ΔClaI that binds to Smads is 4-5-fold lower than that of Fast1 (Chen et al., 1996, Nature 383: 691-696) bound to Smad2 in the same assay (Liu et al., 1997, Genes Dev. 11: 3157-3167). Gli3C′ΔClaI associated with all Smads tested, Smad1, 2, 3, and 4, and the complexes were negatively affected by cell incubation with the appropriate agonist.

To identify regions in Gli3 involved in Smad interactions, Smad-binding activity of Gli3C′ΔClaI, two terminal deletions of Gli3C′ΔClaI which conserve the zinc finger region, and Gli3C′ΔXhoI, which lacks the three C-terminal zinc fingers were tested (see FIG. 6) shown to bind DNA directly¹⁹. Gli3C′ΔClaI and its derived deletions formed complexes with Smad1 and in all cases BMP signaling partially inhibited complex formation. Complex formation, however, was not detected with Gli3C′ΔhoI, indicating that the last three zinc fingers are required for Smad-binding. The inability of Gli3C′ΔXhoI to bind Smads is correlated with its inability to inhibit dorsal mesodermal development.

The result of misexpression of Gli3, and to a lesser Gli2, in early frog embryos is reminiscent of local inhibition of ventral mesodermal patterning produced by injection of dominant-negative BMP receptors (Harland et al., 1997, Ann. Rev. Cell. Dev. Biol. 13: 611-667) or the inhibitory Smad6 (Hata et al., 1998, Genes Dev. 12: 186-197). The result of Gli3 injection is also similar to that with Nodal-related3 (Xnr3) injection, a dorsally expressed TGFβ superfamily member (Smith et al., 1995, Cell 82: 37-46), suggesting that Gli2 and Gli3 can affect both dorsal and ventral mesoderm, promoting an intermediate state. hn normal development, full-length Gli2 and Gli3 in ventrolateral mesoderm could therefore modify the character of mesoderm induced by BMPs, whereas C-terminally truncated repressor forms could modify dorsalization by Activins or Nodals secreted by dorsal cells. These results also raise the possibility that Shh/Gli1, which are expressed in the organizer (Lee et al., Development 124: 2537-2552; Ruiz I Altaba et al., 1995, Mol. Cell. Neurosci. 6: 106-121), cooperate with Activins and/or Nodals in dorsal mesodermal development. Gli2/3 may thus not directly inhibit TGFβ family signaling but rather modify its outcome at downstream steps. This is consistent with the inability of full-length proteins to interact with Smads and the inability of the repressor form of Gli3 to affect transcriptional activity from the BMP-inducible Vent2 enhancer (Candia et al., 1997, Development 124: 4467-4480), the TGFβ-inducible Mix2 promoter (Huang et al., 1995, EMBO J. 14: 5965-5973 or by Gal4-Smad1 or Gal4-Smad2 from a Gal4 reporter gene (Liu et al., 1997, Genes Dev. 11: 3157-3167). It remains possible, however, that the Gli3 repressor-Smad complex has novel binding or transcriptional specificities.

In the neural tube, ventrally produced Shh antagonizes the dorsalizing effects of BMP (Liem et al., 1995, Cell 82: 969-979. One possibility is that full-length Gli3 modifies the result of BMP signaling whereas repressor forms inhibit Gli1, and thus Shh, function. BMP-induced dissociation of Smad-Gli repressor complexes would induce a two tier antagonism of the Shh pathway. On one hand, Smads would be free to bind specific partners to induce BMP-responsive dorsal gene expression. One the other hand, the free repressor forms of Gli3 would act to antagonize ventralizing Shh/Gli1 function, as well as that of activating Gli3 forms. In addition, complex formation is likely to occur in the cytoplasm where inactive Smads normally reside (Massagu, 1998, Ann. Rev. Biochem. 67: 753-791), suggesting that depending on the relative abundance of each protein, Smads could render Gli2/3 repressors inactive by cytoplasmic sequestration until signaling occurs. The regulation of the production of repressor forms from full-length proteins would thus appear critical for determining the signaling outcome.

The complex functional interactions between Gli and Smad signaling pathways that can be inferred from the results in vivo are mirrored by the complex set of physical interactions between Gli and Smad proteins observed in transfected cells in vitro. These studies uncover novel molecular mechanisms the consequence of which is just beginning to be understood. Indeed, integration of Hedgehog and BMP signaling is likely to occur at different levels and here suggest an integration at the level of Gli⅔ repressor-Smad interaction. In addition, Glis and Smads may integrate multiple signaling inputs as in the case of Smad1 which integrates BMP and EGF signaling (Kretzschmar et al., 1997, Nature 389: 618-622).

The findings also have important implications for the understanding of two human diseases, Polydactyly type A (PAP-A) and Pallister-Hall syndrome (PHS) that arise from the production of C-terminally truncated Gli3 proteins (Kang et al., 1997, Nature Genet. 15: 266-268; Radhakrishna et al., 1997, Nature Genetics 17: 269-271). PAP-A involves that development of an extra posterior sixth digit whereas PHS is a pleiotropic lethal syndrome affecting many organs and tissues. The PAP-A and PHS truncated Gli3 proteins approximate in vivo processed forms predicted to act as dominant repressors. The results suggest that in addition to an inhibition of all Gli function in regions that express Gli3, the pathology of PAP-A and PHS involves modification of TGFβ family signaling.

### EXAMPLE 3

Sonic Hedgehog Regulates the Development of the Cerebellum

How the verebrate brain develops remains one of the greatest challenges in embryology. The cerebellum, or little brain, has been intensely studied in part due to its structural simplicity, the existence of mutants affecting its function and the fact that it, as the cerebral neocortex, develops in a layered fashion. However, the molecular bases of cerebellar development have remained largely unknown. Here it is shown that Sonic hedgehog (Shh) signaling is involved in the development of the cerebellum. SHH controls both the proliferation of granule neurons, the most abundant cell type in the brain, and the differentiation of Bergmann radial glia, which are known to provide differentiation factors for granule neurons. Together, these and previous findings allow the formulation of a model for the coordinate regulation of cerebellar development in which SHH-producing Purkinje neurons play a central role. In addition, the findings provide a cellular context for medulloblastomas, childhood cancers of the cerebellum.

The vertebrate cerebellum has been the focus of intense study for over a century and consists of deep nuclei and a large cortical region. The cortex shows species-specific foliation patterns and distinct cell types are positioned in a layered fashion. The outer most layer, the external germinal layer (EGL) contains dividing granule neuron progenitors. Post-mitotic granule cells migrate inwards, guided by Bergmann radial glial fibers and past the Purkinje neuron layer (PL), to form the internal granular layer (IGL) where granule neurons terminally differentiate. Analysis of cerebellar granule cell development (Hatten and Heinz, 1995, Ann. Rev. Neurosci. 18: 385-408; Smeyne et al., 1995, Mol. Cell. Neurosci. 6: 230-251; Hatten et al., 1997, Current Opinion Neurobiol. 7: 4047; Herrup and Kuemerle, 1997, Ann. Rev. Neurosci. 20: 61-90) have shown that EGL cells acquire a granule neuron fate from the time of their migration from the rhombic lip and that EGL proliferation requires cell contact. Later born Bergmann glia have been shown to require a secreted factor for their maturation and then induce the differentiation of EGL cells to become mature granule neurons (Hatten, 1985, J. Cell. Biol. 100: 384-396; Gao et al., 1991, Neuron 6: 705-715; Hunter et al., 1995, PNAS 92: 2061-2065; Adler et al., 1996, Neuron 17: 389-399 Jankovski et al., 1996, Europ. J. Neurosci. 8: 2308-2319; Soriano et al., 1997, Neuron 18: 563-577). In addition, there are mutual interactions between granule and Purkinje neurons required for the normal development of both cell types (Hatten and Heinz, 1995, Ann. Rev. Neurosci. 18: 385-408). These studies, however, have not defined the identity of the factors involved in their proliferation or differentiation although several peptide growth factors, including Insulin-like and Epidermal growth factors, can potentiate granule neuron precursor proliferation (Gao et al., 1991, Neuron 6: 705-715).

Renewed interest in the molecular basis of cerebellar development stems from the finding that cerebellar cancers, or medulloblastomas (MBs), form in mice and humans lacking Patched (Ptc) function (Hahn et al., 1996, Cell 85: 841-851; Hahn et al. 1998, Nature Medicine 4: 619-622; Johnson et al., 199 Science 272: 1668-1671; Goodrich et al., 1997, Science 277: 1109-1113). Shh is a conserved signaling molecule that is thought to bind the membrane receptor Ptc and inhibit its repression of Smoothened (Smo). Smo then transduces the Shh signal intracellularly, which is normally inhibited by protein kinase A (PKA), leading to the nuclear action of Gli family transcription factors (Ingham, 1998, Curr.Op. Gen. Dev. 8: 88-94, Ruiz i Altaba, 1997, Cell 90: 193-196). This signaling pathway is required for the patterning of many embryonic tissues including the ventral neural tube (Mart' et al., 1995 Nature 375: 322-325; Chiang et al., 1996, Nature 383: 407-413; Ericson et al., 1996, Cell 87:661-673). In add MBs, deregulated Shh signaling leading to Gli1 expression causes two other types of familial and sporadic tumors: basal cell carcinomas (BCCs) in the skin and rhabdomyosarcomas in muscle (Hahn et al., 1996, Cell 85: 841-851; Hahn et al., 1998, Nature Med. 4: 619-622; Johnson et al., 1996, Science 272: 1668-1671; Dahmane et al., 1997, Nature 389: 876-881; Oro et al., 1997, Science 276: 817-821; Xie et al., 1998, Nature 391: 90-92; Ingham, 1998, Curr. Op. Gen. Dev. 8: 88-94). BCCs recapitulate follicular differentiation (Ackerman et al., 1993, Neoplasms with Follicular Differentiation, Lea and Febinger; Dahmane et al., 1997, Nature 389: 876-881) and MBs may recapitulate ontogenic steps of cerebellar external germinal cells (Trojanowski et al., 1992Mol. Chem. Neuropath. 17: 121-135). The latter would be consistent with the expression of Ptc and Gli genes in the rodent cerebellum (Millen et al., 1995, Development 121: 3935-3945; Goodrich et al., 1996, Science 277: 1109-1113; Traiffort et al., 1998, J. Neurochemistry 70: 1327-1330). Specifically, Ptc is expressed by granule neuron precursors in the EGL and later on in cells of the Purkinje cell layer, possibly Bergmann glia (Goodrich et al., 1996; Science 277: 1109-1113; Traiffort et al., 1998, J. Neurochemistry 70: 1327-1330), raising the possibility that Shh signaling affects these cell types. The idea that MBs recapitulate steps of granule neuron development is also supported by the finding that the majority of human MBs show expression of ZIC1 (Yokota et al., 1996, Cancer Res. 56: 377-383), a zinc finger protein of the GLI superfamily, and other genes expressed in granule neurons or their precursors in the EGL (Aruga et al., 1994, J. Neurochem. 63: 1880-1890; Kozmik et al., 1995, PNAS 92: 5709-5713). Nevertheless, it is not known whether endogenous Shh signaling plays a role in normal cerebellar development.

Here, the idea that MBs represent a perverted granule cell progenitor state to include an endogenous role for SHH in cerebellar development is extended. SHH is endogenously produced by Purkinje neurons and also transiently by early EGL cells. The results of SHH-treatment and those obtained with a blocking anti-SHH antibody show the requirement of SHH in the proliferation of granule neuron precursors. In addition, glial differentiation is induced by SHH secreted by Purkinje neurons. Thus, these results provide the molecular basis for the central role of Purkinje neurons in the regulation of proliferation and differentiation of the cerebellar cortex.

### Materials and Methods

Animals, explant preparation and cell isolation: Embryonic days for mice (E) or chicks (D) were counted starting as 0.5 the morning after conception for mice or the days of incubation for chicks. Mice were obtained from Taconic or Charles River and fertilized chick eggs from Spafas. Embryos or post-natal (P) mice were dissected in L15-air on ice. EGL cerebellar explants were prepared from D10-12 chicks by first isolating the cortical region, which contained the PL with small amounts of IGL, with a surgical scapel and then cutting this into squares. The lateral-most regions were avoided. 50-100 explants were routinely obtained from a single D10 cerebellum. Explants were grown in collagen gels (Tessier-Lavigne et al., 1988; Nature 336: 775-778) with serum-free media (Nothias et al., 1998; Curr. Biol. 8: 459-462). Granule neuron precursors and glia were isolated by a percoll gradient (Hatten, 1985, J. Cell Biol. 100: 384-396; Gao et al., 1991, Neuron 6: 705-715) followed by sequential platings. Isolated neuronal precursors were allowed to aggregate for 24 h and isolated glia were treated after 2 days in culture in serum-free media.

In situ hybridization and immunofluorescence: In situ hybridization was carried out on cryostat sections of fixed or perfused speciments with 4% paraformaldehyde (Dahmane et al., 1997, Nature 389: 876-881). Antisense digoxygenin labelled RNA probes for mouse Gli, Zic, Shh genes were as described (Dahmane et al., 1997, Nature 389: 876-881; Aruga et al., 1994, J. Neurochem. 63: 1880-1890; 1996a, J. Biol. Chem. 271: 1043-1047). Mouse probes were also successfully used for chick tissue given the high conservation of these genes. In situ hybridization sections were counterstained with hematoxylin (Sigma) to localize transcript expression.

Immnunocytochemistry was carried out in 4% parafolmaldehyde-fixed/perfused tissue with anti-SHH mAb (5E1; Ericson et al., 1996) obtained from the University of Iowa Hybridoma Bank; anti-BLBP polyclonal antibody (Feng et al., 1994, Neuron 12: 895-908); anti-GFAP polyclonal antibodies (Sigma); anti-Calbindin 28K polyclonal antibodies (Sigma); anti-ZIC proteins mAb (Yokota et al., 1996, Cancer Res. 56: 377-383); anti-TAG polyclonal antibodies (Furley et al., 1990, Cell 61: 157-170); TuJ1 anti-neural tubulin mAb (Babco); anti-HNF-3β mAb (Ruiz i Altaba, 1994, J. Dev. Biol. 40: 1081-1088; Ericson et al., 1996, Cell 87: 661-673); and anti-BrdU mnAb (Becton-Dickinson or Harlan-seralab). Primary antibody labeling was visualized with fluorescein or rhodamine labeled secondary antibodies (Boehringer Mannheim, TAGO) Blocking antibodies against SHH (5E1; Ericson et al., 1996, Cell 87: 661-673) were used at 5 μg/ml.

Growth factors and chemicals: Baculovirus-derived purified SHH (SHH-N) was used at 5 ng/ml. Forskolin and 1,9dideoxyforskolin (Sigma) were used at 10 mM. BrdU (Sigma) was used at 6 μg/ml for a 1 hr pulse 24 h before harvesting the explants. HCl treatment and visualization of BrdU-labelled DNA was done following standard protocols. DAPI (Sigma) was used at 10 ng/ml to counterstain DNA in nuclei.

### Results

Expression of SHH and Shh-responsive genes in the developing cerebellum: In the mouse cerebellum, expression of Shh mRNA was detected in EGL and immature Purkinje cells at E19 a time when Gli1 was also found in the PL and EGL (Millen et al., 1995, Development 121: 3935-3945). At P2, their expression in the EGL became stronger. At P5, when some EGL cells have already migrated inwards passing through the PL to form the IGL, Shh was no longer detected in EGL cells and was confined to Purkinje neurons. Gli1, in contrast, maintained expression in the EGL and PL (Millen et al., 1995, Development 121: 3935-3945). Gli2 and Gli3 were expressed also in the EGL and PL although the level of Gli3 was very low overall and that of Gli2 was weak in the PL (Millen et al., 1995, Development 121: 3935-3945). Expression of Zic1-3 was found at high levels in the IGL, as previously reported (Aruga et al., 1996a, J. Biol. Chem. 271: 1043-1047; 1996b, Gene 171: 291-294), but also in EGL cells. Within the EGL, Gli1 was expressed in dividing progenitors in the outer layer (oEGL), Gli2 was expressed throughout the EGL and Zic1 was predominantly expressed in postmitotic pre-migratory cells of the inner layer (iEGL). Taking Gli1 and Gli2 expression as a marker of response to Shh signaling (Marigo et al., 1996, Dev. Biol. 180: 273-283; Lee et al., 1997, Development 124: 2537-2552; Ruiz i Altaba, 1998, Development 125: 2203-2212), SHH could thus act autocrinely in early EGL cells. SHH secreted from Purkinje neurons could then act on oEGL cells and in cells within the PL, possibly Bergmann glia and/or in an autocrine fashion.

The distribution of SHH protein and its localization with respect to a variety of cell-specific markers as prelude for functional studies was carried out in the chick cerebellum as the pattern of cerebellar cell types is highly conserved. At D9 SHH protein was found in immature Purkinje neurons migrating away from the ventricular zone, where they are born, towards the EGL. At this time, glia identified by expression of Glial fibrilary acidic protein (GFAP) or Brain lipid-binding protein (BLBP, Feng et al., 1994, Neuron 12: 895-908) were located close to the ventricular zone where thay are also born. By D11, Purkinje neurons expressing SHH were adjacent to GFAP+ Bergmann glia, close to the proliferating EGL and at D15, Purkinje neurons and Bergmann glia were at their final neighboring positions. This was also seen in P5 mice as detected by expression of the Purkinje neuron marker Calbindin and that of BLBP in Bergmann glia. At D15 SHH protein is detected in Purkinje neurons. Co-expression of Calbindin and SHH showed that the levels of SHH protein decreased between D15 and the time of hatching (D21). Post-mitotic pre-migratory cells of the iEGL were marked by TAG1 expression (Furley et al., 1990, Cell 61: 157-170; Kuhar et al., 1993, Development 117: 97-104), and IGL as well as a few migrating cells in the molecular layer were marked by the expression of ZIC proteins (Yokota et al., 1996, Cancer Res. 56: 377-383), which are required for normal development (Aruga et al., 1998, J. Neurosci. 18: 284-293). Moderate HNF-3β expression in the IGL, providing a plausible basis for the balance defects observed in HNF-3β+/− mice (Weinstein et al., 1994, Cell 78: 575-588). Expression of SHH, Gli1 and HNF-3β in adjacent cells of the developing cerebellum constitutes a parallel with their expression in the notochord and ventral neural tube (Roelink et al., 1994, Cell 76: 761-775; Ruiz i Altaba et al., 1995, Mol. Cell. Neurosci. 6: 106-121; Lee et al., 1997, Development 124: 2537-2552) and raises the possibility that Shh signaling induces the differentiation of both floor plate cells and granule neurons.

SHH enhances expression of Gli1 and Zic1 in cortical cerebellar explants: To investigate of the role of SHH in cerebellar development an in vitro explant assay was developed in which the EGL and adjacent PL, with little IGL, are dissected and grown in collagen gels in serum-free media. These cerebellar cortical explants showed the expression of all markers tested except those of Purkinje neurons, consistent with the difficulty to grow these neurons in vitro (Baptista et al., 1994, Neuron 12: 243-260; Dusart et al., 1997, Neuroscience 17: 3710-3726). This suggests that there is no endogenous source of SHH in the explant cultures although it is important to note that explanted EGL cells are likely to have been already exposed to SHH before explantation. In situ hybridization of sectioned DII explants grown for 72 h revealed low levels of Gli1 and Zic1 expression, which in chick embryos were found to be expressed in both EGL and IGL. SHH treatment induced higher expression of these markers, indicating an enhancement of the development of granule neurons by Shh signaling.

SHH induces spreading and proliferation of and neurite outgrowth from granule neurons in cortical explants: D12 explants treated with SHH for 24, 48 or 96 h showed increased dispersion of cells into the surrounding collagen gel. This migratory behavior was further revealed by the identification of dividing cells as judged by incorporation of BrdU. Spreading of cells into the surrounding collagen matrix was not observed at 48 h in control explants although there was some spreading at 96 h. The assay can thus identify cells with migratory behavior, recapitulating the normal migration of differentiating EGL cells towards the IGL, across the PL. Control untreated explants grown for 48h showed very few BrdU labeled cells outside the bulk of the explant but some TAG+ neurites were observed. SHH treatment greatly increased the number of BrdU labeled cells and the number of TAG+ neurites growing into the collagen matrix. Confocal imaging showed, however, that only a small fraction (5-10%) of BrdU+ cells were also TAG+.

Since TAG is normally expressed in a transient manner in iEGL cells becoming granule neurons, it seemed possible that the majority of spreading BrdU+ cells corresponded to ZIC+ IGL granule neurons. SHH treatment for 48 h induced the migration of large numbers of ZIC+ cells into the collagen, not seen in control explants. Confocal imaging revealed that the majority (85-90%) of BrdU+ spreading cells were ZIC+ as were those inside the explant. Together, these results show that in explants, SHH induces the progression of granule neurons through three sequential steps characteristic of the oEGL, iEGL and IGL, respectively: i- proliferation, ii- upregulation of TAG expression and iii) expression of ZIC proteins in post-mitotic granule neurons that have migrated into the collagen matrix.

SHH directly induces proliferation of purified granule neuronal progenitors: The effects of SHH on explant culture could be direct on granule neuron progenitors or indirect through an intermediate cell type. To test whether SHH is a mitogen for granule neuron precursors, these cells were isolated through a percoll gradient (Hatten, 1985, Ann. Rev. Neuroscience 18: 385-408) from P1-3 mice cerebella and allowed to form aggregates in vitro. Such neuronal aggregates do not contain significant glial contamination as revealed by the absence of GFAP labeling, and show a modest degree of proliferation as measured by continuous BrdU incorporation after 24 h in culture (FIG. 2L, M, T; Hatten, 1985, Ann. Rev. Neuroscience 18: 385408; Gao et al., 1991, Neuron 6: 705-715). Addition of SHH to the serum-free culture media for 24 h greatly increased their proliferation by 34 fold, demonstrating a direct effect of SHH on granule cell progenitors and providing a model for the hyperproliferative behavior of medulloblastomas.

Anti-SHH antibodies and forskolin inhibit granule neuron development: Explants treated with a blocking anti-SHH monoclonal antibody (mAb, Ericson et al., 1996, Cell 87: 661-673) failed to show any appreciable spreading of BrdU+ or ZIC+ cells at any time point. Moreover, the scant outgrowth of TAG+ neurites seen in control explants was inhibited by mnAb treatment. In addition, mAb treatment also decreased ZIC immunoreactivity within the explants. Thus, blocking Shh signaling inhibits the migration of and neurite extension from differentiating granule neurons.

The modest decrease in ZIC immunoreactivity in D12 explants could reflect the inability of the mAb to inhibit the proliferation of progenitors that have already been exposed to SHH. The effects of mAb incubation in earlier cerebellar explants. Untreated D10 explants grown for 96 h showed levels of BrdU incorporation similar to those seen in D12 explants were tested. Incubation with mAb, however, decreased BrdU incorporation by 3-4 fold inside the explant and completely inhibited the number of BrdU+ cells spreading into the collagen. Anti-SHH mAb treatment also inhibited the proliferation of purified mouse granule neuron aggregates by 3-4 fold.

Requirement of the Shh signaling pathway was also tested by elevating the intracellular levels of cAMP with forskolin and thus enhancing the activity of PKA, a known inhibitor of this pathway (Fan et al., 1995, Cell 81: 457-465). BrdU incorporation in DIO explants decreased 3-4 fold after treatment for 96 h with forskolin, as compared to that seen in untreated control explants and those treated with 1,9 dideoxyforskolin, an inactive derivative. In addition, isolated mouse granule neuronal precursors treated with forskolin for 24 h exhibited 4-6 fold lower levels of BrdU incorporation than control aggregates. Together, the results strongly suggest that SHH is normally required for proliferation of granule neuron progenitors.

SHH induces the differentiation of Bergmann glia: Double labeling of explants with the pan-neuronal marker TuJ1 and the Bergmann glia marker BLBP showed that SHH induced both neurite outgrowth as well as massive migration of glia with radial morphology from the explant into the collagen matrix which was 10-20 fold greater than that observed in untreated explants. MAb-treated explants showed less neuronal outgrowth than controls and no glial spreading, suggesting that SHH is required for the maturation and/or migration of glia. Single labeling with BLBP or GFAP further confirmed this finding, showing that cells invading the collagen matrix in SHH treated explants had long processes characteristic of radial glia.

To test if SHH induces enhanced glial proliferation, the expression of BLBP in recently divided cells was analyzed. Double labeling experiments with BrdU and BLBP showed that only a very small fraction (3-5%) of BLBP+ cells were also BrdU+ following treatment with SHH for 4 h or 24 h in the continous presence of BrdU. In addition, the majority of spreading BrdU+ cells were located on top of or near BLBP+ processes, suggesting that these represented granule neurons migrating over radial glia (Hatten, 1990, Trends Neurosci. 13: 179-184; Komuro and Rackic, 1998, J. Neurosci. 18: 1478-1490).

To test for a direct effect, astroglia were isolated from P1-3 mice cerebella via a percoll gradient (Hatten, 1985, J. Cell Biol. 100: 384-396) and plated in vitro. Culture of purified glia at low density showed that after 3 days in culture these were BLBP negative, had a flat morphology and readily divided (Hatten, 1985; J. Cell Biol. 100: 384-396). In contrast, treatment with SHH at day 2 for 24 h induced the expression of BLBP in over 90% of the cells, showing that SHH induces their maturation/differentiation. Assessment of the ability of anti-SHH mAb to directly block glial differentiation was difficult as glia do not differentiate in isolation under the conditions used here. However, the results in explants suggest a requirement of SHH in this process.

### Discussion

The results presented here indicate that SHH regulates cerebellar development by acting directly on neighboring populations of neurons and glia. For granule neurons, SHH functions as a mitogen, like in the retina (Jensen and Wallace, 1997, Development 124: 363-371) and unlike its trophic and protective roles for other types of neurons (Miao et al., 1997, J. Neurosci. 17: 5891-5899). In contrast, for Bergmann glia, SHH acts as a differentiation factor, like for spinal oligodendrocytes (Pringle et al., 1996, Dev. Biol. 177: 30-42; Poncet et al., 1996; Mech. of Dev. 60: 13-32). How distinct neurons and glia respond differently to SHH remains unclear although SHH could synergize with other factors, such as FGFs (Ye et al., 1997; Cell 93: 755-766), to elicit distinct responses. The proposed ability of SHH from Purkinje neurons to induce proliferation of granule neuron precursors illustrates how post-mitotic neurons can affect the development of neighboring precursors. This kind of neuron-precursor interaction is likely to be critical in the development of the vertebrate brain, in which there are highly dynamic and interpedendent cellular relationships. For example, post-mitotic neurons have also been shown to induce the proliferation of astrocytes (Fruttiger et al., 1996, Neuron 17: 1117-1131).

The results together with those of previous studies (Hatten and Heinz, 1995, Ann. Rev Neurosci. 18: 385-408; Smeyne et al., 1995, Mol. And Cell. Neurosci. 6: 230-251) suggest a model for the coordinate regulation of cerebellar cell development by SHH. Granule neuronal precursors in the rhombic lip are already committed to this fate, perhaps responding to dorsal neural tube signal (Hatten et al., 1997, Curr.Op. Neurobiol. 7: 40-47) and require SHH signaling for their proliferation. Proliferation of granule neuronal precursors within the EGL may be driven by autocrine SHH signaling first (FIG. 3A) and then by SHH from Purkinje neurons, providing a molecular basis for the requirement of these neurons in the normal development of the EGL and later of granule neurons (Smeyne et al., 1995, Mol. Cell. Neurosci. 6: 230-251; Hatten and Heinz, 1995, Ann. Rev. Neurosci. 18: 385-408; Herrup and Kuemerle, 1997, Ann. Rev. Neurosci. 20: 61-90). The possibility of secretion of SHH protein from dendrites is supported by its inducing action from retinal axons in flies (Huang and Kunes, 1997, Cell 86: 411-422) and the detection of SHH immunoreactivity in axonal tracts in the chick brain. SHH from Purkinje neurons is also proposed to direct the differentiation of later born Bergmann glia. SHH may thus drive a regulatory circuit in which it induces both proliferation of the oEGL and differentiation of iEGL cells into granule neurons, the latter through the intermediate action of a differentiation factor from Bergmann glia. Aspects of maturation and/or differentiation of Purkinje neurons may also be regulated by SHH signaling acting in an autocrine manner.

Involvement of SHH in cerebellar development is also suggested by the finding that distal inhibitors of cholesterol biosynthesis abolish Shh signaling (Incardona et al., 1998, Development, in press; Cooper et al., 1998, Science 280: 1603-1607), as SHH needs to be modified by cholesterol (Porter et al., 1996, Science 274: 255-269), and inhibit cerebellar development in rodents (Repetto et al., 1990, Teratology 42: 611-618; Dehart et al., 1997, Amer. J. Med. Gen. 68: 328-337; Lanuoue et al., 1997, Amer. J. Med. Gen. 73: 24-31). In humans, the Smith-Lemli-Opitz syndrome (SLOS; Gorlin et al., 1990, Syndromes of the Head and Neck, Oxford University Press; Kelly et al., 1996, Am. J. Med. Gen. 66: 478-484) arises from the failure to synthesize cholesterol (Salen et al., 1996, J. Lipid Res. 37: 1169-1180; Farese and Herz, 1998, Trends in Genetics 14: 115-120), phenocopies loss of SHH function (Belloni et al., 1996, Nature Gen. 14: 353-356; Roessler et al., 1996, Nature Gen. 14: 357-360; Chiang et al., 1996, Nature 383: 407-413; Cooper et al., 1998, Science 280: 1603-1607) and SLOS patients display a hypoplastic cerebellum (Ness et al., 1997, Am. J. Med. Gen. 68: 294-299). In contrast, the inability to down regulate the Shh signaling pathway in early EGL cells may underlie the hyperproliferative behavior of MBs. Sporadic MBs could also arise from the failure of Bergmann glia, or other cell types, to provide differentiation factors that antagonize the proliferative effects of Shh signaling. Anti-MB agents are thus likely to include factors that inhibit the function of the Shh signaling pathway, as well as factors that induce the differentiation of MBs into neurons, which could die in the absence of appropriate trophic support.

What is claimed is:

1. An isolated protein comprising an amino acid sequence of a C-terminal Gli 1 truncate, wherein the protein acts as a dominant-negative repressor of neuronal differentiation.

2. The isolated protein of claim 1, wherein the protein is a C-terminal deleted Pstl Gli 1 truncate.

3. The isolated protein of claim 1, wherein the protein is made by cleaving the nucleic acid sequence which codes for an amino acid sequence of a full length Gli 1 protein with a PstI restriction endonuclease, thereby creating a C-terminal deleted Pstl Gli 1 truncate.

4. The isolated protein of claim 1, wherein the protein has the amino acid sequence as set forth in SEQ ID NO:1.

5. The isolated protein of claim 1, wherein the protein is a C-terminal deleted BsaBI Gli 1 truncate.

6. The isolated protein of claim 1, wherein the protein is made by cleaving the nucleic acid sequence which codes for an amino acid sequence of a full length Gli 1 protein with a BsaBI restriction endonuclease, thereby creating a C-terminal deleted BsaBI Gli 1 truncate.

7. The isolated protein of claim 1, wherein the protein has the amino acid sequence as set forth in SEQ ID NO:2.

8. The isolated protein of claim 1, wherein the protein is a C-terminal deleted Agel Gli 1 truncate.

9. The isolated protein of claim 1, wherein the protein is made by cleaving the nucleic acid sequence which codes for an amino acid sequence of a full-length Gli 1 protein with an AgeI restriction endonuclease, thereby creating a C-terminal deleted AgeI Gli 1 truncate.

10. The isolated protein of claim 1, wherein the protein has the amino acid sequence as set forth in SEQ ID NO:3.

11. An isolated protein comprising an amino acid sequence of a C-terminal Gli 3 truncate, wherein the protein acts as a dominant-negative repressor of neuronal differentiation.

12. The isolated protein of claim 11, wherein the protein is a C-terminal deleted XhoI Gli 3 truncate.

13. The isolated protein of claim 11, wherein the protein is made by cleaving the nucleic acid sequence which codes for an amino acid sequence of a full-length Gli 3 protein with an XhoI restriction endonuclease, thereby creating a C-terminal deleted XhoI Gli 3 truncate.

14. The isolated protein of claim 11, wherein the protein has the amino acid sequence as set forth in SEQ ID NO:4.

15. The isolated protein of claim 11, wherein the protein is a C-terminal deleted Bal#8 Gli 3 truncate.

16. The isolated protein of claim 11, wherein the protein is made by cleaving the nucleic acid sequence which codes for an amino acid sequence of a full length Gli 3 protein with a Bal#8 restriction endonuclease, thereby creating a C-terminal deleted Bal#8 Gli 3 truncate.

17. The isolated protein of claim 11, wherein the protein has the amino acid sequence as set forth in SEQ ID NO:5.

18. The isolated protein of claim 11, wherein the protein is a C-terminal deleted ClaI Gli 3 truncate.

19. The isolated protein of claim 11, wherein the protein is made by cleaving the nucleic acid sequence which codes for an amino acid sequence of a full-length Gli 3 protein with a Clal restriction endonuclease, thereby creating a C-terminal deleted ClaI Gli 3 truncate.

20. The isolated protein of claim 11, wherein the protein has the amino acid sequence as set forth in SEQ ID NO:6.

21. An isolated C-terminal Gli 1 truncate protein, wherein the protein comprises 1075-1125 amino acids and acts as a dominant-negative repressor of neuronal differentiation.

22. The isolated protein of claim 21, wherein the amino acid sequence is set forth in SEQ ID NO:7.

23. An isolated C-terminal Gli 1 truncate protein, wherein the protein comprises 735-785 amino acids and acts as a dominant-negative repressor of neuronal differentiation.

24. The isolated protein of claim 23, wherein the amino acid sequence is set forth in SEQ ID NO:8.

25. An isolated C-terminal Gli 1 truncate protein, wherein the protein comprises 515-565 amino acids and acts as a dominant-negative repressor of neuronal differentiation.

26. The isolated protein of claim 25, wherein the amino acid sequence is set forth in SEQ ID NO:9.

27. An isolated C-terminal Gli 3 truncate protein, wherein the protein comprises 975-1025 amino acids and acts as a dominant-negative repressor of neuronal differentiation.

28. The isolated protein of claim 27, wherein the amino acid sequence is set forth in SEQ ID NO:10.

29. An isolated C-terminal Gli 3 truncate protein, wherein the protein comprises 865-915 amino acids and acts as a dominant-negative repressor of neuronal differentiation.

30. The isolated protein of claim 29, wherein the amino acid sequence is set forth in SEQ ID NO:11.

31. An isolated C-terminal Gli 3 truncate protein, wherein the protein comprises 735-785 amino acids and acts as a dominant-negative repressor of neuronal differentiation.

32. The isolated protein of claim 31, wherein the amino acid sequence is set forth in SEQ ID NO:12

33. An isolated C-terminal Gli 3 truncate protein, wherein the protein comprises 775-725 amino acids and acts as a dominant-negative repressor of neuronal differentiation.

34. The isolated protein of claim 33, wherein the amino acid sequence is set forth in SEQ ID NO:13.

35. An isolated C-terminal Gli 3 truncate protein, wherein the protein comprises 620-670 amino acids and acts as a dominant-negative repressor of neuronal differentiation.

36. An isolated analog of the protein of claims 1, 10, 11 and 14.

37. An isolated protein according to claim 36, wherein the analog comprises the amino acid sequence having a N-terminal methionine.

38. An isolated protein according to claim 36, wherein the analog comprises the amino acid sequence having a N-terminal polyhistidine.

39. An isolated nucleic acid fragment which encodes an amino acid sequence of a C-terminal Gli 1 truncate, wherein the protein acts as a dominant-negative repressor of neuronal differentiation.

40. The isolated nucleic acid of claim 39, wherein the nucleic acid is a C-terminal deleted Pstl Gli 1 fragment.

41. The isolated nucleic acid of claim 39, wherein the nucleic acid is a C-terminal deleted BsaBI Gli 1 fragment.

42. The isolated nucleic acid of claim 39, wherein the nucleic acid is a C-terminal deleted AgeI Gli 1 fragment.

43. The isolated nucleic acid of claim 39, wherein the nucleic acid is set forth in SEQ ID NO:15.

44. An isolated nucleic acid fragment which encodes an amino acid sequence of a C-terminal Gli 3 truncate, wherein the protein acts as a dominant-negative repressor of neuronal differentiation.

45. The isolated nucleic acid of claim 44, wherein the nucleic acid is a C-terminal deleted XhoI Gli 3 fragment.

46. The isolated nucleic acid of claim 44, wherein the nucleic acid is a C-terminal deleted Bal#8 Gli 3 fragment.

47. The isolated nucleic acid of claim 44, wherein the nucleic acid is a C-terminal deleted ClaI Gli 3 fragment.

48. The isolated nucleic acid of claim 44, wherein the nucleic acid is set forth in SEQ ID NO:16.

49. The isolated nucleic acid of claim 39, wherein the nucleic acid is DNA.

50. The isolated nucleic acid of claim 39, wherein the nucleic acid is cDNA.

51. The isolated nucleic acid of claim 39, wherein the nucleic acid is genomic DNA.

52. The isolated nucleic acid of claim 39, wherein the nucleic acid is RNA.

53. An isolated nucleic acid of claim 39 operatively linked to a promoter of RNA transcription.

54. A vector which comprises the isolated nucleic acid of claim 39.

55. The vector of claim 54, wherein the promoter comprises a bacterial, yeast, insect or mammalian promoter.

56. The vector of claim 54, wherein the vector is a plasmid, cosmid, yeast artificial chromosome (YAC), bacteriophage or eukaryotic viral DNA.

57. A host vector system for the production of a protein which comprises the vector of claim 54 in a suitable host cell.

58. The host vector system of claim 57, wherein the suitable host cell comprises a prokaryotic or eukaryotic cell.

59. A cell line comprising the isolated nucleic acid of claims 39 or 44.

60. A method of obtaining a protein in purified form which comprises:
(a) introducing the vector of claim 54 into a suitable host cell;
(b) culturing the resulting host cell so as to produce the protein;
(c) recovering the protein produced in step (b); and
(d) purifying the protein so recovered in step (c).

61. The isolated nucleic acid of claims 39 or 44, wherein the isolated nucleic acid has a marker, label or tag.

62. An antibody capable of specifically binding to the protein of claims 1 or 11.

63. The antibody of claim 62, wherein the antibody is a monoclonal antibody.

64. The antibody of claim 62, wherein the antibody is a polyclonal antibody.

65. The antibody of claim 62, wherein the antibody is a chimeric antibody.

66. A pharmaceutical composition comprising an amount of the protein of claims 1, 10, 11 and 14 and a pharmaceutically acceptable carrier or diluent.

67. A pharmaceutical composition comprising an amount of the analog of claim 36 and a pharmaceutically acceptable carrier or diluent.

68. A method of testing the ability of a drug, agent, or compound to modulate the activity of the protein of claims 1, 10, 11 and 14, which comprises:
(a) culturing test cells which contain elevated levels of the protein of claims 1, 10, 11 and 14 in a tumorous condition;
(b) adding the drug, agent, or compound under test; and
(c) measuring the change if any, in the tumorous condition of said test cells.

69. A method for identifying a test composition or agent which modulates the proteins of claims 1, 10, 11 and 14 which comprises:
(a) contacting the protein of claims 1, 10, 11 and 14 with a test composition or agent under conditions permitting binding between the proteins and the test composition;
(b) detecting specific binding of the a test composition or agent to the proteins; and
(c) determining whether the a test composition or agent inhibits the proteins, so as to identify a test composition or agent which is which modulates Gli 3.

70. A method of identifying a test composition or agent which modulates binding to the proteins of claims 1, 10, 11 and 14, the method comprising:
(a) incubating components comprising the test composition, and the proteins, wherein the incubating is carried out under conditions sufficient to permit the components to interact; and
(b) measuring the effect of the test composition on the binding to the proteins.

71. A method of identifying/screening a cell for protein truncates or fragments of Gli protein family comprising, introducing into the cell a protein of claim 1, 10, 11 and 14, wherein the protein inhibits the function or activity of a protein of the Gli family; and detecting the resulting protein produced, thereby identifying/screening the cell for protein truncates or fragments of the Gli protein family.

72. A method of inhibiting the function, or processing of Gli 1 or Gli 3, comprising introducing into a cell the proteins of claim 1, 10, 11 and 14, or the vector of claim 54, thereby inhibiting the function of or processing of Gli 1, Gli 2, or Gli 3.

73. A method for treating a subject having Polydactyly Type A (PAP-A) or Pallister-Hall Syndrome (PHS), comprising administering a therapeutically effective amount of the pharmaceutical composition of claim 65 so as to inhibit the function or processing of Gli 1 or Gli 3 expression, thereby treating the subject having Polydactyly Type A (PAP-A) or Pallister Syndrome (PHS).


SEQUENCE LISTING (16 sequences)

SEQ ID NO: 1 - protein, 515 aa, Xenopus laevis
MASRQCPPAAVFNSMNPPVNSYVEHCYLRSPNVMAEGMNEMPYCHQTNLMTSHHGFGLAQGSDHLAGTDGSRFSTPRSTMKLSKKRAMSISPLSDASIDLQTMIRTSPNSLVAFINSRCSSASGSYGHLSIGTISPSLGYQNCLNHQRPQAGPYGSNPLMPYNSHEHLSSRGMSMLQPRSSVKHCQLKSEPLSITGLDTIGSKRLEDGSEGDISSPASVGTQDPLLGLLDGRDDLEKDDGKHEPETVYETNCHWESCTKEFDTQEHLVHHINNEHIHGEKKEFVCHWQDCSRELRPFKAQYMLVVHMRRHTGEKPHKCTFEGCNKAYSRLENLKTHLRSHTGEKPYVCEHEGCNKAFSNASDRAKHQNRTHSNEKPYVCKIPGCTKRYTDPSSLRKHVKTVHGPEAHITKKHRGDGMLRAQPGHEGPGNQNVKGENQLDMEASSACKEDGRLAVPDITLKSQPSPGGQSSCSSERSPLGSTNNNDSGVEMNANTGGSFEDLTNLDDIPSVDSMGT

SEQ ID NO: 2 - protein, 1253 aa, Xenopus laevis
MASRQCPPAAVFNSMNPPVNSYVEHCYLRSPNVMAEGMNEMPYCHQTNLMTSHHGFGLAQGSDHLAGTDGSRFSTPRSTMKLSKKRAMSISPLSDASIDLQTMIRTSPNSLVAFINSRCSSASGSYGHLSIGTISPSLGYQNCLNHQRPQAGPYGSNPLMPYNSHEHLSSRGMSMLQPRSSVKHCQLKSEPLSITGLDTIGSKRLEDGSEGDISSPASVGTQDPLLGLLDGRDDLEKDDGKHEPETVYETNCHWESCTKEFDTQEHLVHHINNEHIHGEKKEFVCHWQDCSRELRPFKAQYMLVVHMRRHTGEKPHKCTFEGCNKAYSRLENLKTHLRSHTGEKPYVCEHEGCNKAFSNASDRAKHQNRTHSNEKPYVCKIPGCTKRYTDPSSLRKHVKTVHGPEAHITKKHRGDGMLRAQPGHEGPGNQNVKGENQLDMEASSACKEDGRLAVPDITLKSQPSPGGQSSCSSERSPLGSTNNNDSGVEMNANTGGSFEDLTNLDDIPSVDSMGTAGASALRKLENLRIDKLNQLRKTPSSGKMVKLPSIHNSGPQGDMSVVCGPLGMSHNQHGIELPASSHVNHLNDRRNSTTSTMSSAYTVSRRSSVVSPYLPNQRAGDSGNMVDSYDISTDPSGHSNEAVCASGLPGLTPAQQYRLKAKYAAATGGPPPTPLPNMERMNTNNRMAFASSDYRGSAISSLQRRHSSNEYHNYGTGIIHPAQAPGAGIRRASDPARTGGDIQAVPKVQRFKSMTNMNVSMMGRQGTSIQQAYGGSDANLQRHMFSPRPPSITENVFMETAGPDEVCHTKEQGFIQSNEMQHYMNYQGQGSQLTAPNDHMNFNPQIHGLDGQSQNVYSHSQRAISNMHLNAENYSGQSNVSNFNQCQMTAHNQHFQNTRQAYNCANLPVQWNEVSSGTMDNPVQRPNHQIMHQNMSPGNHCQSQLSNCTVPESTKQGCPVNRNSCQQGMYMNNQKYNHGGQVQVKPEQQFHHSAPAMMSCQNMKHPSRQEHHFTKTNTMPLSSEATNCDYQGQQDSTQNSCFNVGLNLNLLSPPGRRSQTPIMQVKEIMVRNYVQSQQALMWEQHPKSMAMMTNSGDDVDTRQNQHKNTLNAAVYMGPKYMNYQGKPSPNNLMSPSSQDSQSSHTKAMGSPSSQCYNFDMMPHPPCGPKPLSRQHSVSSQSTYMGSPNQLSPSYQSSESSPRRMACLPPIQPQSEVTNNTSMMYYTGQMEMHQSKPGVHKLTTPLNLNQTSCD

SEQ ID NO: 3 - protein, 525 aa, Xenopus laevis
MASRQCPPAAVFNSMNPPVNSYVEHCYLRSPNVMAEGMNEMPYCHQTNLMTSHHGFGLAQGSDHLAGTDGSRFSTPRSTMKLSKKRAMSISPLSDASIDLQTMIRTSPNSLVAFINSRCSSASGSYGHLSIGTISPSLGYQNCLNHQRPQAGPYGSNPLMPYNSHEHLSSRGMSMLQPRSSVKHCQLKSEPLSITGLDTIGSKRLEDGSEGDISSPASVGTQDPLLGLLDGRDDLEKDDGKHEPETVYETNCHWESCTKEFDTQEHLVHHINNEHIHGEKKEFVCHWQDCSRELRPFKAQYMLVVHMRRHTGEKPHKCTFEGCNKAYSRLENLKTHLRSHTGEKPYVCEHEGCNKAFSNASDRAKHQNRTHSNEKPYVCKIPGCTKRYTDPSSLRKHVKTVHGPEAHITKKHRGDGMLRAQPGHEGPGNQNVKGENQLDMEASSACKEDGRLAVPDITLKSQPSPGGQSSCSSERSPLGSTNNNDSGVEMNANTGGSFEDLTNLDDIPSVDSMGTAGASALRKLE

SEQ ID NO: 4 - protein, 547 aa, Homo sapiens
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHK

SEQ ID NO: 5 - protein, 700 aa, Homo sapiens
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHKCTFEGCTKAYSRLENLKTHLRSHTGEKPYVCEHEGCNKAFSNASDRAKHQNRTHSNEKPYVCKIPGCTKRYTDPSSLRKHVKTVHGPEAHVTKKQRGDIHPRPPPPRDSGSHSQSRSPGRPTQGALGEQQDLSNTTSKREECLQVKTVKAEKP

SEQ ID NO: 6 - protein, 745 aa, Homo sapiens
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHKCTFEGCTKAYSRLENLKTHLRSHTGEKPYVCEHEGCNKAFSNASDRAKHQNRTHSNEKPYVCKIPGCTKRYTDPSSLRKHVKTVHGPEAHVTKKQRGDIHPRPPPPRDSGSHSQSRSPGRPTQGALGEQQDLSNTTSKREECLQVKTVKAEKPMTSQPSPGGQSSCSSQQSPISNYSNSGLELPLTDGGSIGDLSAID

SEQ ID NO: 7 - protein, 1100 aa, Xenopus laevis
MASRQCPPAAVFNSMNPPVNSYVEHCYLRSPNVMAEGMNEMPYCHQTNLMTSHHGFGLAQGSDHLAGTDGSRFSTPRSTMKLSKKRAMSISPLSDASIDLQTMIRTSPNSLVAFINSRCSSASGSYGHLSIGTISPSLGYQNCLNHQRPQAGPYGSNPLMPYNSHEHLSSRGMSMLQPRSSVKHCQLKSEPLSITGLDTIGSKRLEDGSEGDISSPASVGTQDPLLGLLDGRDDLEKDDGKHEPETVYETNCHWESCTKEFDTQEHLVHHINNEHIHGEKKEFVCHWQDCSRELRPFKAQYMLVVHMRRHTGEKPHKCTFEGCNKAYSRLENLKTHLRSHTGEKPYVCEHEGCNKAFSNASDRAKHQNRTHSNEKPYVCKIPGCTKRYTDPSSLRKHVKTVHGPEAHITKKHRGDGMLRAQPGHEGPGNQNVKGENQLDMEASSACKEDGRLAVPDITLKSQPSPGGQSSCSSERSPLGSTNNNDSGVEMNANTGGSFEDLTNLDDIPSVDSMGTAGASALRKLENLRIDKLNQLRKTPSSGKMVKLPSIHNSGPQGDMSVVCGPLGMSHNQHGIELPASSHVNHLNDRRNSTTSTMSSAYTVSRRSSVVSPYLPNQRAGDSGNMVDSYDISTDPSGHSNEAVCASGLPGLTPAQQYRLKAKYAAATGGPPPTPLPNMERMNTNNRMAFASSDYRGSAISSLQRRHSSNEYHNYGTGIIHPAQAPGAGIRRASDPARTGGDIQAVPKVQRFKSMTNMNVSMMGRQGTSIQQAYGGSDANLQRHMFSPRPPSITENVFMETAGPDEVCHTKEQGFIQSNEMQHYMNYQGQGSQLTAPNDHMNFNPQIHGLDGQSQNVYSHSQRAISNMHLNAENYSGQSNVSNFNQCQMTAHNQHFQNTRQAYNCANLPVQWNEVSSGTMDNPVQRPNHQIMHQNMSPGNHCQSQLSNCTVPESTKQGCPVNRNSCQQGMYMNNQKYNHGGQVQVKPEQQFHHSAPAMMSCQNMKHPSRQEHHFTKTNTMPLSSEATNCDYQGQQDSTQNSCFNVGLNLNLLSPPGRRSQTPIMQVKEIMVRNYVQSQQALMWEQHPKSMAMMTNSGDDVD

SEQ ID NO: 8 - protein, 760 aa, Xenopus laevis
MASRQCPPAAVFNSMNPPVNSYVEHCYLRSPNVMAEGMNEMPYCHQTNLMTSHHGFGLAQGSDHLAGTDGSRFSTPRSTMKLSKKRAMSISPLSDASIDLQTMIRTSPNSLVAFINSRCSSASGSYGHLSIGTISPSLGYQNCLNHQRPQAGPYGSNPLMPYNSHEHLSSRGMSMLQPRSSVKHCQLKSEPLSITGLDTIGSKRLEDGSEGDISSPASVGTQDPLLGLLDGRDDLEKDDGKHEPETVYETNCHWESCTKEFDTQEHLVHHINNEHIHGEKKEFVCHWQDCSRELRPFKAQYMLVVHMRRHTGEKPHKCTFEGCNKAYSRLENLKTHLRSHTGEKPYVCEHEGCNKAFSNASDRAKHQNRTHSNEKPYVCKIPGCTKRYTDPSSLRKHVKTVHGPEAHITKKHRGDGMLRAQPGHEGPGNQNVKGENQLDMEASSACKEDGRLAVPDITLKSQPSPGGQSSCSSERSPLGSTNNNDSGVEMNANTGGSFEDLTNLDDIPSVDSMGTAGASALRKLENLRIDKLNQLRKTPSSGKMVKLPSIHNSGPQGDMSVVCGPLGMSHNQHGIELPASSHVNHLNDRRNSTTSTMSSAYTVSRRSSVVSPYLPNQRAGDSGNMVDSYDISTDPSGHSNEAVCASGLPGLTPAQQYRLKAKYAAATGGPPPTPLPNMERMNTNNRMAFASSDYRGSAISSLQRRHSSNEYHNYGTGIIHPAQAPGAGIRRASDPARTGGDIQAVPKVQRFKSMTNMNVS

SEQ ID NO: 9 - protein, 540 aa, Xenopus laevis
MASRQCPPAAVFNSMNPPVNSYVEHCYLRSPNVMAEGMNEMPYCHQTNLMTSHHGFGLAQGSDHLAGTDGSRFSTPRSTMKLSKKRAMSISPLSDASIDLQTMIRTSPNSLVAFINSRCSSASGSYGHLSIGTISPSLGYQNCLNHQRPQAGPYGSNPLMPYNSHEHLSSRGMSMLQPRSSVKHCQLKSEPLSITGLDTIGSKRLEDGSEGDISSPASVGTQDPLLGLLDGRDDLEKDDGKHEPETVYETNCHWESCTKEFDTQEHLVHHINNEHIHGEKKEFVCHWQDCSRELRPFKAQYMLVVHMRRHTGEKPHKCTFEGCNKAYSRLENLKTHLRSHTGEKPYVCEHEGCNKAFSNASDRAKHQNRTHSNEKPYVCKIPGCTKRYTDPSSLRKHVKTVHGPEAHITKKHRGDGMLRAQPGHEGPGNQNVKGENQLDMEASSACKEDGRLAVPDITLKSQPSPGGQSSCSSERSPLGSTNNNDSGVEMNANTGGSFEDLTNLDDIPSVDSMGTAGASALRKLENLRIDKLNQLRKTPS

SEQ ID NO: 10 - protein, 1000 aa, Homo sapiens
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHKCTFEGCTKAYSRLENLKTHLRSHTGEKPYVCEHEGCNKAFSNASDRAKHQNRTHSNEKPYVCKIPGCTKRYTDPSSLRKHVKTVHGPEAHVTKKQRGDIHPRPPPPRDSGSHSQSRSPGRPTQGALGEQQDLSNTTSKREECLQVKTVKAEKPMTSQPSPGGQSSCSSQQSPISNYSNSGLELPLTDGGSIGDLSAIDETPIMDSTISTATTALALQARRNPAGTKWMEHVKLERLKQVNGMFPRLNPILPPKAPAVSPLIGNGTQSNNTCSLGGPMTLLPGRSDLSGVDVTMLNMLNRRDSSASTISSAYLSSRRSSGISPCFSSRRSSEASQAEGRPQNVSVADSYDPISTDASRRSSEASQSDGLPSLLSLTPAQQYRLKAKYAAATGGPPPTPLPNMERMSLKTRLALLGDALEPGVALPPVHAPRRCSDGGAHGYGRRHLQPHDALGH

SEQ ID NO: 11 - protein, 890 aa, Homo sapiens
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHKCTFEGCTKAYSRLENLKTHLRSHTGEKPYVCEHEGCNKAFSNASDRAKHQNRTHSNEKPYVCKIPGCTKRYTDPSSLRKHVKTVHGPEAHVTKKQRGDIHPRPPPPRDSGSHSQSRSPGRPTQGALGEQQDLSNTTSKREECLQVKTVKAEKPMTSQPSPGGQSSCSSQQSPISNYSNSGLELPLTDGGSIGDLSAIDETPIMDSTISTATTALALQARRNPAGTKWMEHVKLERLKQVNGMFPRLNPILPPKAPAVSPLIGNGTQSNNTCSLGGPMTLLPGRSDLSGVDVTMLNMLNRRDSSASTISSAYLSSRRSSGISPCFSSRRSSEASQAEGRPQNVS

SEQ ID NO: 12 - protein, 760 aa, Homo sapiens
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHKCTFEGCTKAYSRLENLKTHLRSHTGEKPYVCEHEGCNKAFSNASDRAKHQNRTHSNEKPYVCKIPGCTKRYTDPSSLRKHVKTVHGPEAHVTKKQRGDIHPRPPPPRDSGSHSQSRSPGRPTQGALGEQQDLSNTTSKREECLQVKTVKAEKPMTSQPSPGGQSSCSSQQSPISNYSNSGLELPLTDGGSIGDLSAIDETPIMDSTISTATTA

SEQ ID NO: 13 - protein, 750 aa, Homo sapiens
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHKCTFEGCTKAYSRLENLKTHLRSHTGEKPYVCEHEGCNKAFSNASDRAKHQNRTHSNEKPYVCKIPGCTKRYTDPSSLRKHVKTVHGPEAHVTKKQRGDIHPRPPPPRDSGSHSQSRSPGRPTQGALGEQQDLSNTTSKREECLQVKTVKAEKPMTSQPSPGGQSSCSSQQSPISNYSNSGLELPLTDGGSIGDLSAIDETPIM

SEQ ID NO: 14 - protein, 645 aa, Homo sapiens
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHKCTFEGCTKAYSRLENLKTHLRSHTGEKPYVCEHEGCNKAFSNASDRAKHQNRTHSNEKPYVCKIPGCTKRYTDPSSLRKHVKTVHGPEAHVTKKQRGD

SEQ ID NO: 15 - DNA, 1021 bp, Homo sapiens
atggcgtcccgccagtgtccgcccgctgctgtctttaactccatgaaccctcctgtgaacagctatgtggaacactgctacctgaggtctcctaatgttatggcggaagggatgaatgaaatgccctattgccatcagaccaaccttatgaccagtcaccatggatttggattggcacaaggcagcgatcacttggcaggcacagatggatcacgtttctctactcctcgaagtacaatgaaactgagcaaaaagagagcaatgtcgatctccccactgtcagatgccagcattgatctacaaaccatgatcaggacctcccccaattccttggtggcttttattaattctagatgttcttcagctagtgggtcttatggccacctgtctataggaactatcagcccatctcttggttatcaaaattgccttaaccatcagaggccacaagcgggaccctatggaagcaacccactcatgccttacaactcccatgaacacctgtccagcagaggaatgagcatgctccagcctcgcagctcagtcaagcactgtcagttgaagtctgagccattgtcgattactggcttggatacgattgggagcaaaagattagaggatggatccgaaggagatatatctagtcctgcttcagttggaacacaggacccactgcttggattgttggatgggcgtgatgacttggagaaggatgatggcaaacatgagccggaaacagtgtacgagaccaactgtcactgggaaagctgtactaaggagtttgacactcaggaacatcttgtccatcacataaacaatgaacacatccatggggaaaagaaggagtttgtgtgtcactggcaggactgttcgcgagaactcagaccctttaaggcacaatatatgctggtggtccatatgaggaggcacacaggagagaaacctcacaaatgcacatttgagggatgtaacaaggcttattcacgacttgagaacttgaagactcacctgaggtcccata

SEQ ID NO: 16 - DNA, 745 bp, Homo sapiens
atggaggcccagtcccacagctccacgaccactgaaaagaaaaaagttgagaattccatagtgaagtgctccactcgaacagatgtgagcgagaaagccgttgcctccagcaccacttctaatgaggatgaaagtcctggacagacttatcacagagagagaagaaacgcaatcactatgcagccacagaatgtccaggggctcagcaaagtcagtgaggaaccttcaacatcgagtgacgagagggcctcattgatcaagaaagagatccatgggtccctgccacacgtggcggagccctctgtgccgtaccgcgggacggtgtttgccatggaccccaggaatggttacatggagccccactaccaccctcctcatcttttccctgccttccatcctcctgtaccaattgatgccagacatcatgagggccgttaccattacgatccatctccgattcctccattgcatatgacttccgccttatctagtagccctacgtatccggacctgcccttcattaggatctccccacaccggaaccccgctgctgcttccgagtctcccttcagccctccacatccctacattaatccctacatggactatatccgctccttgcacagcagcccatcgctctccatgatctcagcaacccgtgggctgagccctacagatgcgccccatgcaggagtcagcccagcagaatactatcatcagatggccctgctaactggccagcgca